SEQUENCE LISTING (74 sequences)

SEQ ID NO: 1 - protein, 38 aa, Galleria mellonella
KVPVGAIKKGGKAIKTGLGVVGAAGTAHEVYSHIRNRH

SEQ ID NO: 2 - protein, 63 aa, Galleria mellonella
MKLTGLFLMIMAVIALFIDVGQADPKVPVGAIKKGGKAIKTGLGVVGAAGTAHEVYSHIRNRH

SEQ ID NO: 3 - protein, 38 aa, Galleria mellonella; identical to SEQ ID NO: 1

SEQ ID NO: 4 - protein, 63 aa, Galleria mellonella
MKLTGLFLMIMAVVALFISVGQADPKVPVGAIKKGGKAIKTGLGVVGAAGTAHEVYSHIRNRH

SEQ ID NO: 5 - protein, 38 aa, Galleria mellonella
KVPIGAIKKGGKIIKKGLGVIGAAGTAHEVYSHVKNRQ

SEQ ID NO: 6 - protein, 63 aa, Galleria mellonella
MKLTGLFLMIMAVLALFVGAGQADPKVPIGAIKKGGKIIKKGLGVIGAAGTAHEVYSHVKNRQ

SEQ ID NO: 7 - protein, 38 aa, Galleria mellonella
KGIGSALKKGGKIIKGGLGALGAIGTGQQVYEHVQNRQ

SEQ ID NO: 8 - protein, 63 aa, Galleria mellonella
MKFFNLVLMVFAVVALMLNGTNAEPKGIGSALKKGGKIIKGGLGALGAIGTGQQVYEHVQNRQ

SEQ ID NO: 9 - protein, 38 aa, Galleria mellonella
KGIGSALKRGGKIIKGGLGALGAIGTGQQVYEHVQNRQ

SEQ ID NO: 10 - protein, 63 aa, Galleria mellonella
MKFFNLVLMVFAVLALMLNGTNAEPKGIGSALKRGGKIIKGGLGALGAIGTGQQVYEHVQNRQ

SEQ ID NO: 11 - DNA, 114 bp, Galleria mellonella
aaagtgcccgttggtgccataaaaaagggtggtaaagctataaaaacaggtttgggtgtggtaggtgccgcaggtacagcgcacgaagtatacagccacatcaggaacaggcac

SEQ ID NO: 12 - DNA, 192 bp, Galleria mellonella
atgaagctgaccggtttattccttatgattatggcagtgatcgcactgtttatagatgttggtcaagctgaccctaaagtgcccgttggtgccataaaaaagggtggtaaagctataaaaacaggtttgggtgtggtaggtgccgcaggtacagcgcacgaagtatacagccacatcaggaacaggcactaa

SEQ ID NO: 13 - DNA, 115 bp, Galleria mellonella
aaaagtgcccgttggtgccatcaaaaagggtggtaaagctataaaaacaggtttgggtgtggtaggtgccgcaggtacagcgcacgaagtatacagccacatcaggaacaggcac

SEQ ID NO: 14 - DNA, 192 bp, Galleria mellonella
atgaagctgaccggtttattccttatgattatggcagtggtcgcactgtttataagtgttggtcaagctgaccctaaagtgcccgttggtgccatcaaaaagggtggtaaagctataaaaacaggtttgggtgtggtaggtgccgcaggtacagcgcacgaagtatacagccacatcaggaacaggcactaa

SEQ ID NO: 15 - DNA, 114 bp, Galleria mellonella
aaggtgcctattggcgccatcaagaaaggtggcaaaattatcaaaaagggtctgggtgtgataggtgccgcaggcacagcgcacgaggtttacagccacgtcaagaacaggcag

SEQ ID NO: 16 - DNA, 192 bp, Galleria mellonella
atgaaattgaccggcctctttctcatgattatggcggtgctcgcgttgtttgtcggcgctggccaagctgatcctaaggtgcctattggcgccatcaagaaaggtggcaaaattatcaaaaagggtctgggtgtgataggtgccgcaggcacagcgcacgaggtttacagccacgtcaagaacaggcagtaa

SEQ ID NO: 17 - DNA, 114 bp, Galleria mellonella
aaaggcatcggcagcgctctcaagaaaggcggaaaaattattaaaggcggcctgggagcattaggagccatcggtaccggccaacaagtgtacgagcatgttcagaacagacaa

SEQ ID NO: 18 - DNA, 192 bp, Galleria mellonella
atgaagttctttaatctcgtattgatggtattcgccgttgtggctttaatgttaaacggtactaatgctgagcccaaaggcatcggcagcgctctcaagaaaggcggaaaaattattaaaggcggcctgggagcattaggagccatcggtaccggccaacaagtgtacgagcatgttcagaacagacaatga

SEQ ID NO: 19 - DNA, 114 bp, Galleria mellonella
aaaggcatcggcagcgctctcaagagaggcggaaaaattattaaaggtggcctgggagcattaggagccatcggtaccggccaacaagtgtacgagcatgttcagaacagacaa

SEQ ID NO: 20 - DNA, 192 bp, Galleria mellonella
atgaagttcttcaatctcgtattgatggtattcgccgttctggctttaatgttaaacggtactaatgctgagcccaaaggcatcggcagcgctctcaagagaggcggaaaaattattaaaggtggcctgggagcattaggagccatcggtaccggccaacaagtgtacgagcatgttcagaacagacaatga

SEQ ID NO: 21 - protein, 43 aa, Artificial
  Consensus sequence for C4/C5 related peptides
XKXXXXAIKKGGXXIXXXXXXXXXAXTAHXXXXXXXXXXXXXX

SEQ ID NO: 22 - protein, 38 aa, Artificial
  Consensus sequence for D related peptides
KGXGXXXXXGGKXIKXGLXXXGXXXXGXXXYXXXXNXX

SEQ ID NO: 23 - protein, 32 aa, Galleria mellonella
KVPIGAIKKGGKIIKKGLGVIGAAGTAHEVYS

SEQ ID NO: 24 - protein, 32 aa, Galleria mellonella
KGIGSALKKGGKIIKGGLGALGAIGTGQQVYE

SEQ ID NO: 25 - DNA, 373 bp, Galleria mellonella
acttacttttcacttccaaccatgaaattgaccggcctctttctcatgattatggcggtgctcgcgttgtttgtcggcgctggccaagctgatcctaaggtgcctattggcgccatcaagaaaggtggcaaaattatcaaaaagggtctgggtgtgataggtgccgcaggcacagcgcacgaggtttacagccacgtcaagaacaggcagtaatttcttataaaaataagctattattagatagtattgtcatggctgtgatatatattgacataattaatttattaaacttaaaccaaattatttttgtaaaccatattttttcatatattgtaataaattaaataaatattttatacattcctcgtgccga

SEQ ID NO: 26 - DNA, 364 bp, Galleria mellonella
tgaattaaaacctaataaacatgaagttctttaatctcgtattgatggtattcgccgttgtggctttaatgttaaacggtactaatgctgagcccaaaggcatcggcagcgctctcaagaaaggcggaaaaattattaaaggcggcctgggagcattaggagccatcggtaccggccaacaagtgtacgagcatgttcagaacagacaatgatgaattttattcattatgttattagtatttatcatatgacataaaagaaccaatcttcaagtctacacctatttgaaatatatttcgattaattgtaaaacctagataaaaaaggaatttaattatcagtttccagccagttgtctcaaata

SEQ ID NO: 27 - DNA, 364 bp, Galleria mellonella
tgaattaaaacctaataaacatgaagttcttcaatctcgtattgatggtattcgccgttctggctttaatgttaaacggtactaatgctgagcccaaaggcatcggcagcgctctcaagagaggcggaaaaattattaaaggtggcctgggagcattaggagccatcggtaccggccaacaagtgtacgagcatgttcagaacagacaatgatgaattttattcattatgttattagtatttatcatatgacataaaagaaccaatcttcaagtctacacctatttgaaatatatttcgattaattgtaaaacctagataaaaaaggaatttaattaccagtttccagccagttgtctcaaata

SEQ ID NO: 28 - DNA, 245 bp, Galleria mellonella
tcagtgaaaccttcactccttgctatcaaccatgaagctgaccggtttattccttatgattatggcagtgatcgcactgtttatagatgttggtcaagctgaccctaaagtgcccgttggtgccataaaaaagggtggtaaagctataaaaacaggtttgggtgtggtaggtgccgcaggtacagcgcacgaagtatacagccacatcaggaacaggcactaaattcaaagagcagaatattagt

SEQ ID NO: 29 - DNA, 245 bp, Galleria mellonella
tcagtgaaaccttcactccttgctatcaaccatgaagctgaccggtttattccttatgattatggcagtggtcgcactgtttataagtgttggtcaagctgaccctaaagtgcccgttggtgccatcaaaaagggtggtaaagctataaaaacaggtttgggtgtggtaggtgccgcaggtacagcgcacgaagtatacagccacatcaggaacaggcactaaattcaaagagcagaatattagt

SEQ ID NO: 30 - protein, 66 aa, Bombyx mori
MNILKFFFVFIVAMSLVSCSTAAPAKIPIKAIKTVGKAVGKGLRAINIASTANDVFNFLKPKKRKH

SEQ ID NO: 31 - protein, 54 aa, Hyblaea puera
AMSLVSCSTAAPAKIPIKAIKTVGKAVGKGLRAINIASTANDVFNFLKPKKRKH

SEQ ID NO: 32 - protein, 42 aa, Antheraea pernyi
AKIPIKAIKTVGKAVGKGLRAINIASTANDVFNFLEPKKRKH

SEQ ID NO: 33 - protein, 41 aa, Heliothis virescens
GKIPIGAIKKAGKAIGKGLRAVNIASTAHDVYTFFKPKKRH

SEQ ID NO: 34 - protein, 67 aa, Spodoptera litura
MKLTKVFVILIVVVALLVPSEAAPGKIPVKAIKKAGAAIGKGLRAINIASTAHDVYSFFKPKHKKKH

SEQ ID NO: 35 - protein, 67 aa, Spodoptera exigua
MKLTKVFVIVIVVVALLVPSEAAPGKIPVKAIKKAGTAIGKGLRAINIASTAHDVYSFFKPKHKKKH

SEQ ID NO: 36 - protein, 67 aa, Manduca sexta
MKLTSLFIFVIVALSLLFSSTDAAPGKIPVKAIKQAGKVIGKGLRAINIAGTTHDVVSFFRPKKKKH

SEQ ID NO: 37 - protein, 37 aa, Caligo illioneus
RKIPVEAIKKGASRAWRALDLASTAYDIASIFNRKRE

SEQ ID NO: 38 - protein, 40 aa, Caligo illioneus
GKIPVEALKKGAKVAGRAWRALDLASTAYDIAHLFDRKRN

SEQ ID NO: 39 - protein, 41 aa, Caligo illioneus
GKIPINAIRKGAKAVGHGLRALNIASTAHDIASAFHRKRKH

SEQ ID NO: 40 - protein, 68 aa, Galleria mellonella
MRLSIILVVVMMVMAMFVSSGDAAPGKIPVKAIKKGGQIIGKALRGINIASTAHDIISQFKPKKKKNH

SEQ ID NO: 41 - protein, 63 aa, Galleria mellonella
MKLTGLFFMIMAMLALFVGAGQADPKVPIGAIKKGGKIIKKGLGVIGAAGTAHEVYSHVKNRH

SEQ ID NO: 42 - protein, 63 aa, Galleria mellonella
MKLTGLFLMIMAVLALFVGAGQADPKVPIGAIKKGGKIIKKGLGVLGAAGTAHEVYNHVRNRQ

SEQ ID NO: 43 - protein, 64 aa, Galleria mellonella
MKFTGIFFIIMAIIALFIGSNEAAPKVNVNAIKKGGKAIGKGFKVISAASTAHDVYEHIKNRRH

SEQ ID NO: 44 - protein, 65 aa, Bombyx mori
MKVFSFFCVVLAMLVLIMGGTSAAPEPKGIGKIIRKGGKVIKHGLTAIGVGAAGHEVYQDSKNSG

SEQ ID NO: 45 - protein, 65 aa, Bombyx mori
MKVFSLFCVVLAMLVLIMGGTSAAPEPKGIGKIIRKGGKVIKHGLTAIGVGAAGHEVYQDSKNSG

SEQ ID NO: 46 - protein, 65 aa, Bombyx mori
MKVFSIFCVVLAMLVLIMGGTSAAPEPKGIGKIIRKGGKVIKHGLTVIGVGAAGHEVYQESKNSG

SEQ ID NO: 47 - protein, 65 aa, Bombyx mori
MKVFSIFCVVLAMLVLIMGGTSAAPEPKGIGKIIRKGGKVIKHGLTVIGVGAAGHDAYQQSQNSG

SEQ ID NO: 48 - protein, 65 aa, Bombyx mori
MKVFSLFCVVLAMLVLIMGGTSAAPEPKGIGKIIRKGGKIIKHGLTVIGVGAAGHDAYQQSQNSG

SEQ ID NO: 49 - protein, 63 aa, Lonomia obliqua
MKLFNFFIVFFALMALMLGGTNAAPKGAGKAIRKGGKIIKHGLTAIGIIGTGHEVYREAKNQG

SEQ ID NO: 50 - protein, 66 aa, Bombyx mori
MYFLKYFIVVLVALSLMICSGQADPKIPVKSLKKGGKVIAKGFKVLTAAGTAHEVYSHVRNRGNQG

SEQ ID NO: 51 - protein, 66 aa, Bombyx mori
MYFLKYFIVVLVALSLMICSGQADPKIPVKSLKKGGKIIAKGFKVLTAAGTAHEVYSHVRNRGNQG

SEQ ID NO: 52 - protein, 66 aa, Bombyx mori
MDFLKYFIVVLVALSLMVCSGQADPKIPVKSLKKGGKIIAKGFKVLTAAGTAHEVYSHVRNRGNQG

SEQ ID NO: 53 - DNA, 21 bp, Artificial
  Oligonucleotide primer
gctttaccaccctttttgatg

SEQ ID NO: 54 - DNA, 19 bp, Artificial
  Oligonucleotide primer
ggtttgggtgtggtaggtg

SEQ ID NO: 55 - DNA, 21 bp, Artificial
  Oligonucleotide primer
catcaaaaagggtggtaaagc

SEQ ID NO: 56 - DNA, 19 bp, Artificial
  Oligonucleotide primer
cacctaccacacccaaacc

SEQ ID NO: 57 - DNA, 22 bp, Artificial
  Oligonucleotide primer
acagtcgcagtcattctcagtc

SEQ ID NO: 58 - DNA, 24 bp, Artificial
  Oligonucleotide primer
cgtagccaataataatactccaca

SEQ ID NO: 59 - DNA, 20 bp, Artificial
  Oligonucleotide primer
accttcactccttgctatca

SEQ ID NO: 60 - DNA, 20 bp, Artificial
  Oligonucleotide primer
taacttacttttcacttcca

SEQ ID NO: 61 - DNA, 20 bp, Artificial
  Oligonucleotide primer
acttatatatatatatatcg

SEQ ID NO: 62 - DNA, 20 bp, Artificial
  Oligonucleotide primer
aaacttatataaatatatcg

SEQ ID NO: 63 - DNA, 20 bp, Artificial
  Oligonucleotide primer
ccnaarggnatcggnwstgc

SEQ ID NO: 64 - DNA, 20 bp, Artificial
  Oligonucleotide primer
tcrtanacytgytgnccngt

SEQ ID NO: 65 - DNA, 20 bp, Artificial
  Oligonucleotide primer
caagaaaggcggcaaaatta

SEQ ID NO: 66 - DNA, 19 bp, Artificial
  Oligonucleotide primer
accgatggctcctaatgct

SEQ ID NO: 67 - DNA, 20 bp, Artificial
  Oligonucleotide primer
tgaattaaaacctaataaac

SEQ ID NO: 68 - DNA, 19 bp, Artificial
  Oligonucleotide primer
tatttgagacaactggctg

SEQ ID NO: 69 - DNA, 20 bp, Artificial
  Oligonucleotide primer
ctcaagaaaggcggcaaaat

SEQ ID NO: 70 - DNA, 19 bp, Artificial
  Oligonucleotide primer
accgatggctcctaatgct

SEQ ID NO: 71 - DNA, 23 bp, Artificial
  Oligonucleotide primer
ggtcaagccgaccctaaggtgcc

SEQ ID NO: 72 - DNA, 23 bp, Artificial
  Oligonucleotide primer
ggctatatacttcagtgcgctgt

SEQ ID NO: 73 - DNA, 21 bp, Artificial
  Oligonucleotide primer
atagtcgagaaatggcaaaat

SEQ ID NO: 74 - DNA, 21 bp, Artificial
  Oligonucleotide primer
ctgcgctatcggcatacacta

(19) United States
(12) Patent Application Publication
(10) Pub. No.: US 2013/0219532 A1
(43) Pub. Date: Aug. 22, 2013

(54) PEPTIDES WITH ANTIFUNGAL ACTIVITIES

(71) Applicants: Peter David East, O'Connor (AU); Susan Elizabeth Brown, Lyneham (AU)

(72) Inventors: Peter David East, O'Connor (AU); Susan Elizabeth Brown, Lyneham (AU)

(73) Assignee: Commonwealth Scientific and Industrial Research Organisation, Campbell (AU)

(21) Appl. No.: 13/851,782

(22) Filed: Mar. 27, 2013

Related U.S. Application Data
(63) Continuation of application No. 12/450,420, filed on Apr. 26, 2010, now abandoned.

(30) Foreign Application Priority Data
Mar. 26, 2007 (AU) 2007901600

(51) Int. Cl. C12N 15/82; A01G 1/00; C07K 14/435

(52) U.S. Cl. CPC C12N 15/8282; C07K 14/43563; A01G 1/001
USPC 800/14; 530/324; 536/23.5; 536/24.31; 435/320.1; 435/419; 435/69.1; 530/387.9; 514/2.4; 800/301; 514/3.3; 47/58.1 R

(57) ABSTRACT

The present invention relates to antifungal and/or antibacterial peptides, especially antifungal peptides obtained from insect species, particularly lepidopterans. The present invention also provides methods of using these antifungal peptides to treat or prevent fungal growth for a variety of purposes, such as protecting plants from fungal infections; treating fungal infections of animals, especially humans; and prevention of food spoilage.

### FIELD OF THE INVENTION

The present invention relates to antifungal and/or antibacterial peptides, especially antifungal peptides obtained from insect species, particularly lepidopterans. The present invention also provides methods of using these antifungal peptides to treat or prevent fungal growth for a variety of purposes such as; protecting plants from fungal infections, treating fungal infections of animals, especially humans, and prevention of food spoilage.

### BACKGROUND OF THE INVENTION

Fungi are eukaryotic cells that may reproduce sexually or asexually and may be biphasic, with one form in nature and a different form in the infected host. Fungal infections of plants and animals are a significant problem in the fields of agriculture, medicine and food production/storage. Fungal infections are becoming a major concern for a number of reasons, including the limited number of antifungal agents available, the increasing incidence of species resistant to older antifungal agents, and the growing population of immunocompromised patients at risk for opportunistic fungal infections.

Fungal diseases of humans are referred to as mycoses. Some mycoses are endemic, where infection is acquired in the geographic area that is the natural habitat of that fungus. These endemic mycoses are usually self-limited and minimally symptomatic. Some mycoses are chiefly opportunistic, occurring in immunocompromised patients such as organ transplant patients, cancer patients undergoing chemotherapy, burn patients, AIDS patients, or patients with diabetic ketoacidosis.

Fungi cause many diseases of plants such as, but not limited to, mildews, rots, rusts, smuts, and wilts etc. For example, soilborne fungal phytopathogens cause enormous economic losses in the agricultural and horticultural industries. In particular, Rhizoctonia solani is one of the major fungal phytopathogens exhibiting strong pathogenicity; it is associated with seedling diseases as well as foliar diseases such as seed rot, root rot, damping-off, leaf and stem rot of many plant species and varieties, resulting in enormous economic losses. Another example is Phytophthora capsici which is a widespread and highly destructive soilborne fungal phytopathogen that causes root and crown rot as well as the aerial blight of leaves, fruit, and the stems of peppers (Capsicum annuum L.).

Plant fungus infection is a particular problem in damp climates and may become a major concern during crop storage. Plants have developed a certain degree of natural resistance to pathogenic fungi; however, modern growing methods, harvesting and storage systems frequently provide a favorable environment for plant pathogens.

Antifungal agents include polyene derivatives, such as amphotericin B and the structurally related compounds nystatin and pimaricin. Furthermore, antifungal peptides have been isolated from a variety of naturally occurring sources (DeLucca and Walsh, 1999). However, there is a need for the identification of further compounds with antifungal activity for use in medical, agricultural and industrial related applications to control and/or prevent fungal growth.

### SUMMARY OF THE INVENTION

The present inventors have previously identified that members of the moricin peptide family possess antifungal activity (WO 2005/080423). These previous studies included a detailed analysis of the Galleria mellonella peptidome to identify Galleria moricin peptides. However, the present inventors have surprisingly identified yet further Galleria mellonella moricin peptides which are structurally distinct from previously described moricin related peptides.

Thus, in a first aspect the present invention provides a substantially purified peptide which comprises a sequence selected from the group consisting of:

i) an amino acid sequence as provided in SEQ ID NO:1 and SEQ ID NO:3,

ii) an amino acid sequence which is at least 85% identical to SEQ ID NO:1 and/or SEQ ID NO:3,

iii) an amino acid sequence as provided in SEQ NO:5,

iv) an amino acid sequence which is at least 98% identical to SEQ ID NO:5,

v) an amino acid sequence as provided in SEQ ID NO:7 or SEQ ID NO:9,

vi) an amino acid sequence which is at least 64% identical to SEQ ID NO:7 and/or SEQ ID NO:9,

vii) a biologically active fragment of any one of i) to vi), and

viii) a precursor comprising the amino acid sequence according to any one of i) to vii),

wherein the peptide, or fragment thereof, has antifungal and/or antibacterial activity.

In a preferred embodiment of the first aspect, the peptide is, where relevant, at least 65%, more preferably at least 70%, more preferably at least 75%, more preferably at least 80%, more preferably at least 85%, more preferably at least 90%, more preferably at least 92%, more preferably at least 95%, more preferably at least 97%, and even more preferably at least 99% identical to the sequence provided in SEQ ID NO:1, SEQ ID NO:3, SEQ ID NO:5, SEQ ID NO:7 and/or SEQ ID NO:9.

Preferably, the precursor of SEQ ID NO:1 is SEQ NO:2, the precursor of SEQ ID NO:3 is SEQ ID NO:4, the precursor of SEQ ID NO:5 is SEQ ID NO:6, the precursor of SEQ ID NO:7 is SEQ ID NO:8, and the precursor of SEQ 113 NO:9 is SEQ ID NO:10.

Preferably, the peptide can be purified from an insect. More preferably, the peptide can be purified from a lepidopteran insect. More preferably, the peptide can be purified from a lepidopteran insect of the family Pyralidae. More preferably, the peptide can be purified from a Galleria sp. Even more preferably, the peptide can be purified from Galleria mellonella.

In a particularly preferred embodiment, the peptide can be purified from an insect which has been exposed to a fungal or bacterial infection. In the case of lepidpoterans, it is preferred that the peptide can be purified from last instar larvae that have been exposed to bacteria such as, but not limited to, Escherichia coli and/or Micrococcus luteus.

In another embodiment, it is preferred that the peptide has a molecular weight of between about 4.5 kDa to about 3.3 kDa. More preferably, the peptide has a molecular weight of about 3.9, or about 3.8 kDa.

In yet a further preferred embodiment, the peptide comprises an N-terminal amphipathic (at least relative to the C-terminus) region which includes a helical structure, a C-terminal hydrophobic (at least relative to the N-terminus) region which also includes a helical structure and an acidic residue, and a charged C-terminal tail.

In a further preferred embodiment, the peptide which is at least 85% identical to SEQ ID NO:1 and SEQ ID NO:3 comprises the amino acid sequence;

(SEQ ID NO: 21)
Xaa₁ Lys Xaa₂ Xaa₃ Xaa₄ Xaa₅ Ala Ile Lys Lys Gly
Gly Xaa₆ Xaa₇ Ile Xaa₈ Xaa₉ Xaa₁₀ Xaa₁₁ Xaa₁₂
Xaa₁₃ Xaa₁₄ Xaa₁₅ Xaa₁₆ Ala Xaa₁₇ Thr Ala His
Xaa₁₈ Xaa₁₉ Xaa₂₀ Xaa₂₁ Xaa₂₂ Xaa₂₃ Xaa₂₄ Xaa₂₅
Xaa₂₆ Xaa₂₇ Xaa₂₈ Xaa₂₉ Xaa₃₀ Xaa₃₁.

Preferably, Xaa₁ is Gly, Pro, Ala or absent, more preferably Gly or absent.

Preferably, Xaa₂ is Ile, Val, Ala, Leu, Met or Phe, more preferably Ile or Val.

Preferably, Xaa₃ is Pro, Gly, Asn, Gln or His, more preferably Pro or Asn.

Preferably, Xaa₄ is Ile, Val, Ala, Leu, Met or Phe, more preferably Ile or Val.

Preferably, Xaa₅ is Lys, Arg, Gly, Pro, Ala, Asn, Gln or His, more preferably Lys, Gly or Asn.

Preferably, Xaa₆ is Gln, Asn, His, Lys or Arg, more preferably Gln or Lys.

Preferably, Xaa₇ is Ile, Val, Ala, Leu or Gly, more preferably Ile or Ala.

Preferably, Xaa₈ is Gly, Pro, Ala, Lys or Arg, more preferably Gly or Lys.

Preferably, Xaa₉ is Thr or Ser, more preferably Thr.

Preferably, Xaa₁₀ is Val, Leu, Ile, Gly, Pro or Ala, more preferably Ala or Gly.

Preferably, Xaa₁₁ is Ile, Vat, Met, Ala, Phe or Leu, more preferably Leu or Phe.

Preferably, Xaa₁₂ is Arg, Lys, Gly, Pro or Ala, more preferably Arg, Gly or Lys.

Preferably, Xaa₁₃ is Gly, Pro, Ala, Val, Ile, Leu, Met, or Phe, more preferably Gly or Val.

Preferably, Xaa₁₄ is Ile, Leu, Val, Ala, Met or Phe, more preferably Val, Ile or Leu.

Preferably, Xaa₁₅ is Asn, Gln, His, Gly, Pro, Ala, Ser or Thr, more preferably Asn, Gly or Ser.

Preferably, Xaa₁₆ is Ile, Val, Ala, Leu or Gly, more preferably Ile or Ala.

Preferably, Xaa₁₇ is Ser, Thr, Gly, Pro or Ala, more preferably Ser or Gly.

Preferably, Xaa₁₈ is Asp or Glu.

Preferably, Xaa₁₉ is Ile, Leu, Val, Ala, Met or Phe, more preferably Ile or Val.

Preferably, Xaa₂₀ is Ile, Leu, Val, Ala, Tyr, Trp or Phe, more preferably Ile or Tyr.

Preferably, Xaa₂₁ is Ser, Thr, Asn, Gln, His, Glu or Asp, more preferably Ser, Asn or Glu.

Preferably, Xaa₂₂ is Gln, Asn or His, more preferably Gln or His.

Preferably, Xaa₂₃ is Phe, Leu, Val, Ala, Ile or Met, more preferably Phe, Val or Ile.

Preferably, Xaa₂₄ is Lys or Arg.

Preferably, Xaa₂₅ is Pro, Gly, Asn, Gln or His, more preferably Pro or Asn.

Preferably, Xaa₂₆ is Lys or Arg.

Preferably, Xaa₂₇ is Lys, Arg, His, Asn or Gln, more preferably Lys, His, Gln or Arg.

Preferably, Xaa₂₈ is Lys, Arg, His, Asn, Gln or absent, more preferably Lys, His or absent.

Preferably, Xaa₂₉ is Lys, Arg or absent, more preferably Lys or absent.

Preferably, Xaa₃₀ is Asn, Gln, His or absent, more preferably Asn or absent.

Preferably, Xaa₃₁ is His, Asn, Gln or absent, more preferably His or absent.

In a further preferred embodiment, the peptide which is at least 64% identical to SEQ ID NO:7 and/or SEQ ID NO:9 comprises the amino acid sequence;

(SEQ ID NO: 22)
Lys Gly Xaa₁ Gly Xaa₂ Xaa₃ Xaa₄ Xaa₅ Xaa₆ Gly Gly
Lys Xaa₇ Ile Lys Xaa₈ Gly Leu Xaa₉ Xaa₁₀ Xaa₁₁ Gly
Xaa₁₂ Xaa₁₃ Xaa₁₄ Xaa₁₅ Gly Xaa₁₆ Xaa₁₇ Xaa₁₈ Tyr
Xaa₁₉ Xaa₂₀ Xaa₂₁ Xaa₂₂ Asn Xaa₂₃ Xaa₂₄.

Preferably, Xaa₁ is Ile, Val, Ala, Leu, or Gly, more preferably Ile.

Preferably, Xaa₂ is Ser, Lys, Thr or Arg, more preferably Ser.

Preferably, Xaa₃ is Ala, Ile, Leu, Val or Gly, more preferably Ala.

Preferably, Xaa₄ is Ile, Val, Ala, Leu, Met or Phe, more preferably Leu.

Preferably, Xaa₅ is Lys or Arg, more preferably Lys.

Preferably, Xaa₆ is Lys or Arg.

Preferably, Xaa₇ is Ile, Val, Leu, Ala, Met or Phe, more preferably Ile.

Preferably, Xaa₈ is Gly, His, Ala, Pro, Asn or Gln, more preferably Gly.

Preferably, Xaa₉ is Gly, Thr, Ala, Pro or Ser, more preferably Gly.

Preferably, Xaa₁₀ is Ala, Val, Leu, Ile, Gly, Met or Phe, more preferably Ala.

Preferably, Xaa₁₁ is Ile, Val, Met, Ala, Phe or Leu, more preferably Leu.

Preferably, Xaa₁₂ is Ala, Val, Ile, Leu, Val, Gly, Met or Phe, more preferably Ala.

Preferably, Xaa₁₃ is Ile, Gly, Pro, Ala, Val or Leu, more preferably Ile.

Preferably, Xaa₁₄ is Gly, Ala, Pro, Val, Leu or Ile, more preferably Gly.

Preferably, Xaa₁₅ is Thr, Ala, Ser, Val, Leu, Ile or Gly, more preferably Thr.

Preferably, Xaa₁₆ is Gln, His or Asn, more preferably Gln.

Preferably, Xaa₁₇ is Gln, Glu, Asp, Asn or His, more preferably Gln.

Preferably, Xaa₁₈ is Ala, Val, Leu, Ile, Gly, Met or Phe, more preferably Val.

Preferably, Xaa₁₉ is Glu, Gln, Arg, Asp, Asn, His or Lys, more preferably Glu.

Preferably, Xaa₂₀ is His, Asp, Glu, Gln or Asn, more preferably His.

Preferably, Xaa₂₁ is Val, Ser, Ala, Thr, Ile, Leu, Met, Phe or Gly, more preferably Val.

Preferably, Xaa₂₂ is Gln, Lys, Asn, His or Arg, more preferably Gln.

Preferably, Xaa₂₃ is Arg, Ser, Gln, Lys, Thr, Are or His, more preferably Arg.

Preferably, Xaa₂₄ is Gln, Gly, Asn, His, Ala or Pro, more preferably Gln.

Preferably, the peptide (or fragment thereof) has antifungal activity. More preferably, the peptide has antifungal activity against the Family of fungi selected from, but not limited to, the group consisting of: Neciriaceae, Pleosporaceae, Mycosphaerellaceae, Phyllachoraceae, Leptosphateria, and Trichocornaceae. More preferably, the peptide has antifungal activity against the Genera of fungi selected from, but not limited to, the group consisting of: Fusarium (also known in the art as Gibberella), Alternaria, Ascochyta, Colletotrichum, Leptosphaerin and Aspergillus. In a particularly preferred embodiment, the peptide has antifungal activity against the Genera of fungi which infect plants selected from, but not limited to, the group consisting of: Alternaria; Ascochyta; Botrytis; Cercospora; Colletotrichum; Diplodia; Erysiphe; Fusarium; Gaeumanomyces; Helminthosporium; Leptosphaeria, Macrophomina; Nectria; Peronospora; Phoma; Phymatotrichum; Phytophthora; Plasmopara; Podosphaera; Puccinia; Puthium; Pyrenophora; Pyricularia; Pythium; Rhizoctonia; Scerotium; Sclerotinia; Septoria; Thielaviopsis; Uncinula; Venturia; and Verticillium. In a further preferred embodiment, the peptide has antifungal activity against the fungi selected from the group consisting of Fusarium graminearum, Fusarium oxysporum, Ascochyta rabid and Leptosphaeria maculans.

In a further aspect, the present invention provides a peptide according to the invention which is fused to at least one other polypeptide/peptide sequence.

In a preferred embodiment, the at least one other polypeptide/peptide is selected from the group consisting of: a polypeptide/peptide that enhances the stability of a peptide of the present invention, a polypeptide/peptide that assists in the purification of the fusion protein, a polypeptide/peptide which assists in the peptide of the invention being secreted from a cell (particularly a plant cell), and a polypeptide/peptide which renders the fusion protein non-toxic to a fungus or a bacteria but which can be processed, for example by proteolytic cleavage, to produce an antifungal peptide of the invention.

In another aspect, the present invention provides an isolated polynucleotide, the polynucleotide comprising a sequence selected from the group consisting of:

i) a sequence of nucleotides provided in any one of SEQ ID NO's 11 to 20;

ii) a sequence encoding a peptide of the invention;

iii) a sequence of nucleotides which is at least 85% identical to at least one of SEQ ID NO's 11 to 14;

iv) a sequence of nucleotides which is at least 98% identical to SEQ ID NO:15 and/or SEQ ID NO:16;

v) a sequence of nucleotides which is at least 64% identical to at least one of SEQ ID NO's 17 to 20; and

vi) a sequence which hybridizes to any one of (i) to (v) under high stringency conditions.

Preferably, the polynucleotide encodes a peptide with antifungal and/or antibacterial activity.

In a preferred embodiment, the polynucleotide is, if relevant, at least 65%, more preferably at least 70%, more preferably at least 75%, more preferably at least 80%, more preferably at least 85%, more preferably at least 90%, more preferably at least 92%, more preferably at least 95%, more preferably at least 97%, and even more preferably at least 99° A) identical to at least one of SEQ ID NO's 11 to 20.

Preferably, the polynucleotide can be isolated from an insect. More preferably, the polynucleotide can be isolated from a lepidopteran insect. More preferably, the polynucleotide can be isolated from lepidopteran insect of the family Pyralidae. More preferably, the polynucleotide can be isolated from a Gafferia sp. Even more preferably, the polynucleotide can be isolated from Galleria mellonella.

In another embodiment, the polynucleotide comprises a sequence provided as SEQ ID NO:11, SEQ ID NO:13, SEQ ID NO:15, SEQ ID NO:17 or SEQ ID NO:19.

Furthermore, the present invention provides a suitable vector for the replication and/or expression of a polynucleotide according to the invention. Thus, also provided is a vector comprising a polynucleotide of the invention.

The vectors may be, for example, a plasmid, virus, transposon or phage vector provided with an origin of replication, and preferably a promotor for the expression of the polynucleotide and optionally a regulator of the promotor. The vector may contain one or more selectable markers, for example an ampicillin resistance gene in the case of a bacterial plasmid or a neomycin resistance gene for a mammalian expression vector. The vector may be used in vitro, for example for the production of RNA or used to transfect or transform a host cell.

In another aspect the present invention provides a host cell comprising a vector, or polynucleotide, of the invention.

Preferably, the host cell is an animal, yeast, bacterial or plant cell. More preferably, host cell is a plant cell.

In a further aspect, the present invention provides a process for preparing a peptide according to the first aspect, the process comprising cultivating a host cell according to the invention under conditions which allow expression of the polynucleotide encoding the peptide, and recovering the expressed peptide.

The present invention also provides peptides produced by a process of the invention.

Also provided is an antibody which specifically binds a peptide of the first aspect. Such antibodies will be useful as markers for peptide production from transgenic systems such as transgenic plants. In addition, such antibodies may be useful in methods of purifying peptides of the invention from insect lysates and/or recombinant expression systems.

In a further aspect, the present invention provides a composition comprising a peptide, a polynucleotide, a vector, an antibody or a host cell of the invention, and one or more acceptable carriers.

In an embodiment, the carrier is a pharmaceutically, veterinary or agriculturally acceptable carrier.

In yet another aspect, the present invention provides a method for killing, or inhibiting the growth and/or reproduction of a fungus, the method comprising exposing the fungus to a peptide of the invention.

As the skilled addressee would be aware, the fungus can be exposed to the peptide by any means known in the art. In one embodiment, the fungus is exposed to a composition comprising the peptide. In another embodiment, the fungus is exposed to a host cell producing the peptide.

Plants and non-human animals resistant to fungal infections can be produced by introducing a polynucleotide of the invention into the plant or animal such that the peptide is produced in the transgenic organism.

Accordingly, in another aspect, the present invention provides a transgenic plant, the plant having been transformed with a polynucleotide according to the present invention, wherein the plant produces a peptide of the invention.

The transgenic plant can be any species of plant, however, it is preferred that the plant is a crop plant. Examples of such crop plants include, but are not limited to, wheat, barley, rice, chickpeas, field peas and the like.

As the skilled person will appreciate, the transgenic plant of the invention may have been directly transformed with the polynucleotide, or be the progeny of a plant that was directly transformed. More specifically, transformed is used to indicate that the polynucleotide is exogenous to the plant.

In a further aspect, the present invention provides a method of controlling fungal infections of a crop, the method comprising cultivating a crop of transgenic plants of the invention.

In addition, in another aspect, the present invention provides a transgenic non-human animal, the animal having been transformed with a polynucleotide according to the present invention, wherein the animal produces a peptide of the invention.

In a further aspect, the present invention provides a method of treating or preventing a fungal infection in a patient, the method comprising administering to the patient a peptide of the invention.

In addition, the present invention provides for the use of a peptide of the invention for the manufacture of a medicament for treating or preventing a fungal infection in a patient.

It is envisaged by the present inventors that the peptides of the invention also has antibacterial activity. Thus, the present invention also provides a method for killing, or inhibiting the growth and/or reproduction of a bacteria, the method comprising exposing the bacteria to a peptide of the invention.

The bacteria can be gram-positive or gram-negative.

As the skilled addressee would be aware, the bacteria can be exposed to the peptide by any means known in the art. In one embodiment, the bacteria is exposed to a composition comprising the peptide. In another embodiment, the bacteria is exposed to a host cell producing the peptide.

In a further aspect, the present invention provides a method of controlling bacterial infections of a crop, the method comprising cultivating a crop of transgenic plants of the invention.

In a further aspect, the present invention provides a method of treating or preventing a bacterial infection in a patient, the method comprising administering to the patient a peptide of the invention.

In addition, the present invention provides for the use of a peptide of the invention for the manufacture of a medicament for treating or preventing a bacterial infection in a patient.

Also provided is a kit comprising a peptide of the invention, a polynucleotide of the invention, a vector of the invention, a host cell of the invention, an antibody of the invention and/or a composition of the invention.

The present inventors are the first to identify that peptides related to Galleria mellonella moricinD possess antifungal activity such as moricin B1 to B8 from Bombyx mori (SEQ ID NO's 44 to 48). Thus, in a further aspect, the present invention provides a method for killing, or inhibiting the growth and/or reproduction of a fungus, the method comprising exposing the fungus to a peptide which comprises a sequence selected from the group consisting of:

i) an amino acid sequence comprising residues 28 to 65 of any one of SEQ ID NO's 44 to 48,

ii) an amino acid sequence comprising residues 26 to 63 of SEQ ID NO:49,

iii) an amino acid sequence comprising residues 26 to 66 of any one of SEQ ID NO's 50 to 52,

iv) an amino acid sequence which is at least 50% identical to any one of i) to iii), and

v) a biologically active fragment of any one of i) to iv).

In yet a further aspect, the present invention provides a method of controlling fungal infections of a crop, the method comprising cultivating a crop of transgenic plants which produce a peptide which comprises a sequence selected from the group consisting of:

i) an amino acid sequence comprising residues 28 to 65 of any one of SEQ ID NO's 44 to 48,

ii) an amino acid sequence comprising residues 26 to 63 of SEQ ID NO:49,

iii) an amino acid sequence comprising residues 26 to 66 of any one of SEQ ID NO's 50 to 52,

iv) an amino acid sequence which is at least 50% identical to any one of i) to iii), and

v) a biologically active fragment of any one of i) to iv).

In yet another aspect, the present invention provides a method of treating or preventing a fungal infection in a patient, the method comprising administering to the patient a peptide which comprises a sequence selected from the group consisting of:

i) an amino acid sequence comprising residues 28 to 65 of any one of SEQ ID NO's 44 to 48,

ii) an amino acid sequence comprising residues 26 to 63 of SEQ ID NO:49,

iii) an amino acid sequence comprising residues 26 to 66 of any one of SEQ ID NO's 50 to 52,

iv) an amino acid sequence which is at least 50% identical to any one of i) to iii), and

v) a biologically active fragment of any one of i) to iv).

Also provided is the use of a peptide which comprises a sequence selected from the group consisting of:

i) an amino acid sequence comprising residues 28 to 65 of any one of SEQ ID NO's 44 to 48,

ii) an amino acid sequence comprising residues 26 to 63 of SEQ ID NO:49,

iii) an amino acid sequence comprising residues 26 to 66 of any one of SEQ ID NO's 50 to 52,

iv) an amino acid sequence which is at least 50% identical to any one of i) to iii), and

v) a biologically active fragment of any one of i) to iv)

for the manufacture of a medicament for treating or preventing a fungal infection in a patient

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. Nucleotide sequence and deduced pre-pro protein sequence of the G. mellonella Gm-moricinC3 gene obtained by PCR on the cDNA library (SEQ ID NO's 25 and 6, respectively). The deduced protein sequence commences at the first in-frame methionine residue. The presumptive secretion signal peptide is shown in italics and the mature Gm-moricinC3 peptide is highlighted in bold font. The peptide sequence obtained by Edman sequencing of the purified Gm-moricinC3 peptide is shown underlined (SEQ ID NO:23). The predicted site of signal peptide cleavage (SignalP) is indicated below the peptide sequence by a single arrow and the predicted site of cleavage to generate the form of the peptide is indicated by a pair of arrows.

FIG. 2. Sequence alignment of the nucleotide sequences of the two Gm-moricinD genes obtained by PCR on the cDNA library (Gm-moricinD—SEQ ID NO:26; Gm-moricinD1—SEQ ID NO:27). The start and stop codons are shown in bold. Non conserved nucleotides are underlined, with mutations resulting in amino acid substitutions in Gm-moricinD1 indicated by a double underline.

FIG. 3. Sequence alignment of the deduced protein sequences of two Gm-moricinD genes obtained by PCR on the cDNA library cDNA clones (SEQ ID NO: 8 and 10). Non conserved residues in the variant Gm-moricinD1 are underlined. The starting amino acid of the mature peptide determined by Edman degradation is indicated in bold.

FIG. 4. Nucleotide sequence and deduced pre-pro protein sequence of the G. mellonella Gm-moricinD gene obtained by PCR on the cDNA library (SEQ ID NO's 26 and 8, respectively). The deduced protein sequence commences at the first in-frame methionine residue. The presumptive secretion signal peptide is shown in italics and the mature Gm-moricinD peptide is highlighted in bold font. The peptide sequence obtained by Edman sequencing of the purified Gm-moricinD peptide is shown underlined (SEQ ID NO:24). The predicted site of signal peptide cleavage (SignalP) is indicated below the peptide sequence by a single arrow and the predicted site of cleavage to generate the mature form of the peptide is indicated by a pair of arrows.

FIG. 5, Sequence alignment of the nucleotide sequences of Gm-moricinC4 (SEQ ID NO:28) and Gm-moricinC5 (SEQ ID NO:29) obtained by PCR on the cDNA library. The start and stop codons are shown in bold. The nucleotides in the open reading frame of Gm-moricinC5 that differ to Gm-moricinC4 are underlined, with mutations resulting in amino acid substitutions indicated by a double underline.

FIG. 6, Sequence alignment of the deduced protein sequences of Gm-moricinC4 (SEQ ID NO:2) and Gm-moricinC5 (SEQ ID NO:4) genes obtained by PCR on the cDNA library cDNA clones. Non conserved residues are underlined. The predicted starting amino acids of the mature peptides are indicated in bold.

FIG. 7. ClustalW alignment of the antifungal peptides from G. mellonella with moricins from other Lepidoptera. G. mellonella (for Gm-A, B, C1 and C2 see WO 2005/080423: Gm-C3, C4, C5 and D disclosed herein), Bombyx mori (Bm-A1—NP_{—}001036829, Bm-A2—CH391671, Bm-A3—AADK01025872, Bm-A4—AV402493, Bm-B1 and Bm-B2—CH380045, Bm-B3, B6 and B8—CH380569), Spodoptera litura (Sl, BAC79440), Spodoptera exigua (Se, AAT38873), Manduca sexta (Ms, AAO74637), Hellothis virescens (Hv, P83416), Hyblaea puera (Hp, AAW21268), Caligo illioneus (Ci-P1646, Ci-P1647, Ci-P1648), Lonomla obligua (translation of CX816233), Antheraea pernyi (Ap, ABF69030).

### KEY TO THE SEQUENCE LISTING

SEQ ID NO:1—Gm-moricinC4 from Galleria mellonella.

SEQ ID NO:2—Pre-Gm-moricinC4 from Galleria mellonella.

SEQ ID NO:3—Gm-moricinC5 from Galleria mellonella.

SEQ ID NO:4—Pre-Gm-moricinC5 from Galleria mellonella.

SEQ ID NO:5—Gm-moricinC3 from Galleria mellonella.

SEQ ID NO:6—Pre-Gm-moricinC3 from Galleria mellonella.

SEQ ID NO:7—Gm-moricinD from Galleria mellonella.

SEQ ID NO:8—Pre-Gm-moricinD from Galleria mellonella.

SEQ ID NO:9—Variant (D1) of Gm-moricinD from Galleria mellonella

SEQ ID NO:10—Variant (D1) of pre-Gm-moricinD from Galleria mellonella

SEQ ID NO:11—cDNA encoding Gm-moricinC4 from Galleria mellonella.

SEQ ID NO:12—cDNA encoding pre-Gm-moricinC4 from Galleria mellonella.

SEQ ID NO:13—cDNA encoding Gm-moricinC5 from Galleria mellonella

SEQ ID NO:14—cDNA encoding pre-Gm-moricinC5 from Galleria mellonella.

SEQ ID NO:15—cDNA encoding Gm-moricinC3 from Galleria mellonella.

SEQ ID NO:16—cDNA encoding pre-Gm-moricinC3 from Galleria mellonella.

SEQ ID NO:17—cDNA encoding Gm-moricinD from Galleria mellonella.

SEQ ID NO:18—cDNA encoding pre-Gm-moricinD from Galleria mellonella.

SEQ ID NO:19—cDNA encoding variant (D1) of Gm-moricinD from Galleria mellonella.

SEQ ID NO:20—cDNA encoding variant (D1) of pre-Gm-moricinD from Galleria mellonella.

SEQ ID NO:21—Consensus sequence for Gm-moricin C4 and GM-moricin C5 related antifungal peptides.

SEQ ID NO:22—Consensus sequence for Gm-moricinD related antifungal peptides.

SEQ ID NO:23—Partial sequence of Gm-moricinC3 purified from Galleria mellonella.

SEQ ID NO:24—Partial sequence of Gm-moricinD purified from Galleria mellonella.

SEQ ID NO:25—Full length cDNA encoding Gm-moricinC3 from Galleria mellonella.

SEQ ID NO:26—Full length cDNA encoding Gm-moricinD from Galleria mellonella.

SEQ ID NO:27—Full length cDNA encoding Gm-moricinD variant (D1) from Galleria mellonella.

SEQ ID NO:28—Full length cDNA encoding Gm-moricinC4 from Galleria mellonella

SEQ ID NO:29—Full length cDNA encoding Gm-moricinC5 from Galleria mellonella

SEQ ID NO:30—Bombyx mori pre-moricin A1.

SEQ ID NO:31—Hyblaea puera moricin.

SEQ ID NO:32—Antheraea pernyl moricin.

SEQ ID NO:33—Heliothis virescens moricin.

SEQ ID NO:34—Spodoptera litura pre-moricin.

SEQ ID NO:35—Spodoptera exigua pre-moricin.

SEQ ID NO:36—Manduca sexta pre-moricin.

SEQ ID NO:37—Caligo illioneus moricin C1-P1647.

SEQ ID NO:38—Caligo illioneus moricin C1-P1648.

SEQ ID NO:39—Caligo illioneus moricin C1-P1646.

SEQ ID NO:40—Galleria mellonella pre-moricin B.

SEQ ID NO:41—Galleria mellonella pre-moricin C1.

SEQ ID NO:42—Galleria mellonella pre-moricin C2.

SEQ ID NO:43—Galleria mellonella pre-moricin A.

SEQ ID NO:44—Bombyx mori pre-moricin B3.

SEQ ID NO:45—Bombyx mori pre-moricin B6.

SEQ ID NO:46—Bombyx mori pre-moricin B2.

SEQ ID NO:47—Bombyx mori pre-moricin B8.

SEQ ID NO:48—Bombyx mori pre-moricin B1.

SEQ ID NO:49—Lorromia obliqua pre-moricin.

SEQ ID NO:50—Bombyx mori pre-moricin A4.

SEQ ID NO:51—Bombyx mori pre-moricin A3.

SEQ ID NO:52—Bombyx mori pre-moricin A1.

SEQ ID NO's 53 to 74—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, microbiology, molecular genetics, immunology, immunohistochemistry, protein chemistry, mycology and biochemistry).

Unless otherwise indicated, the recombinant protein, cell culture, transgenic plant production and microbiological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), and are incorporated herein by reference.

As used herein, the term “antifungal” peptide refers to a peptide having antifungal properties, e.g., which inhibits the growth of fungal cells, or which kills fungal cells, or which disrupts or retards stages of the fungal life cycle such as spore germination, sporulation, and mating.

As used herein, the term “antibacterial” peptide refers to a peptide having antibacterial properties, e.g., which inhibits the growth of bacterial cells, or which kills bacterial cells, or which disrupts or retards stages of the bacteria life cycle such as spore formation, and cell division.

### Polypeotides/Peptides

By “substantially purified peptide” or “purified peptide” we mean a peptide that has generally been separated from the lipids, nucleic acids, other peptides, and other contaminating molecules with which it is associated, in its native state. Preferably, the substantially purified peptide or purified peptide is at least 60% free, more preferably at least 75% free, and more preferably at least 90% free from other components with which it is naturally associated.

The terms “polypeptide” and “peptide” are generally used interchangeably. However, the term “peptide” is typically used to refer to chains of amino acids which are not large, for instance 100 or less residues in length.

The % identity of a peptide is determined by GAP (Needleman and Wunsch, 1970) analysis (GCG program) with a gap creation penalty=8, and a gap extension penalty-3. The query sequence is at least 15 amino acids in length, and the GAP analysis aligns the two sequences over a region of at least 15 amino acids. More preferably, the query sequence is at least 50 amino acids in length, and the GAP analysis aligns the two sequences over a region of at least 50 amino acids. Preferably, the GAP analysis aligns the two sequences over their entire length.

As used herein a “biologically active” fragment is a portion of a peptide of the invention which maintains a defined activity of the full length peptide. In most embodiments this activity is antifungal activity, however, in some embodiments this activity is antibacterial. Biologically active fragments can be any size as long as they maintain the defined activity, however, in a preferred embodiment they are at least 10, more preferably at least 15, amino acids in length.

Amino acid sequence mutants of the peptides of the present invention, can be prepared by introducing appropriate nucleotide changes into a nucleic acid of the present invention, or by in vitro synthesis of the desired peptide. Such mutants include, for example, deletions, insertions or substitutions of residues within the amino acid sequence. A combination of deletion, insertion and substitution can be made to arrive at the final construct, provided that the final peptide product possesses the desired characteristics.

Mutant (altered) peptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes related to those of the present invention, such as that encoding moricin from B. mori (Hare and Yamakawa, 1995). Peptide products derived from mutated/altered DNA can readily be screened using techniques described herein to determine if they possess antifungal and/or antibacterial activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the peptide molecule removed and a different residue inserted in its place. The sites of greatest interest for substitutional mutagenesis include sites identified as the active site(s). Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

In particular, it has previously been shown that moricin possesses two α-helical structures (Hemmi et al., 2002). Considering the relatedness of the peptides of the invention to moricin-like peptides (see FIG. 7), it is possible that a similar structure is also important for maintaining antifungal activity of the peptides of the invention. Accordingly, when designing mutants of, for example, SEQ ID NO:1 the skilled addressee, using knowledge of the chemistry of particular amino acids combined with known methods of predicting peptide tertiary structure, can readily produce peptides with one or a few amino acid variations when compared to SEQ ID NO:1 which possess antifungal activity.

Furthermore, if desired, unnatural amino acids or chemical amino acid analogues can be introduced as a substitution or addition into the peptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are peptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the peptide of the invention.

Peptides of the present invention can be produced in a variety of ways, including production and recovery of natural peptides, production and recovery of recombinant peptides, and chemical synthesis of the peptides. In one embodiment, an isolated peptide of the present invention is produced by culturing a cell capable of expressing the peptide under conditions effective to produce the peptide, and recovering the peptide. A preferred cell to culture is a recombinant cell of the present invention. Effective culture conditions include, but are not limited to, effective media, bioreactor, temperature, pH and oxygen conditions that permit peptide production. An effective medium refers to any medium in which a cell is cultured to produce a peptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

By “isolated polynucleotide” we mean a polynucleotide which has generally been separated from the polynucleotide sequences with which it is associated or linked in its native state. Preferably, the isolated polynucleotide is at least 60% free, more preferably at least 75% free, and more preferably at least 90% free from other components with which it is naturally associated. Furthermore, the term “polynucleotide” is used interchangeably herein with the term “nucleic acid molecule”.

The % identity of a polynucleotide is determined by GAP (Needleman and Wunsch, 1970) analysis (GCG program) with a gap creation penalty=8, and a gap extension penalty=3. The query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. Preferably, the GAP analysis aligns the two sequences over their entire length.

A polynucleotide of the present invention may selectively hybridise to a polynucleotide that encodes a peptide of the present invention under high stringency. Furthermore, oligonucleotides of the present invention have a sequence that hybridizes selectively under high stringency to a polynucleotide of the present invention. As used herein, high stringency conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis or DNA shuffling on the nucleic acid as described above). It is thus apparent that polynucleotides of the invention can be either naturally occurring or recombinant.

Oligonucleotides of the present invention can be RNA, DNA, or derivatives of either. The minimum size of such oligonucleotides is the size required for the formation of a stable hybrid between an oligonucleotide and a complementary sequence on a nucleic acid molecule of the present invention. The present invention includes oligonucleotides that can be used as, for example, probes to identify nucleic acid molecules, or primers to amplify nucleic acid molecules of the invention.

### Recombinant Vectors

One embodiment of the present invention includes a recombinant vector, which comprises at least one isolated polynucleotide molecule of the present invention, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises a polynucleotide molecule of the present invention operatively linked to an expression vector. The phrase operatively linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in recombinant cells of the present invention, including in bacterial, fungal, endoparasite, arthropod, animal, and plant cells. Particularly preferred expression vectors of the present invention can direct gene expression in plants cells. Vectors of the invention can also be used to produce the peptide in a cell-free expression system, such systems are well known in the art.

In particular, expression vectors of the present invention contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod and mammalian cells, such as, but not limited to, tac, lac, trp, Ur, oxy-pro, omp/Ipp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intenuediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells. Particularly preferred transcription control sequences are promoters active in directing transcription in plants, either constitutively or stage and/or tissue specific, depending on the use of the plant or parts thereof. These plant promoters include, but are not limited to, promoters showing constitutive expression, such as the 35S promoter of Cauliflower Mosaic Virus (CaMV), those for leaf-specific expression, such as the promoter of the ribulose bisphosphate carboxylase small subunit gene, those for root-specific expression, such as the promoter from the glutamine synthase gene, those for seed-specific expression, such as the cruciferin A promoter from Brassica napus, those for tuber-specific expression, such as the class-I palmist promoter from potato or those for fruit-specific expression, such as the polygalacturonase (PG) promoter from tomato.

Recombinant molecules of the present invention may also (a) contain secretory signals (i.e., signal segment nucleic acid sequences) to enable an expressed peptide of the present invention to be secreted from the cell that produces the peptide and/or (b) contain fusion sequences which lead to the expression of nucleic acid molecules of the present invention as fusion proteins. Examples of suitable signal segments include any signal segment capable of directing the secretion of a peptide of the present invention. Preferred signal segments include, but are not limited to, tissue plasminogen activator (t-PA), interferon, interleukin, growth hormone, viral envelope glycoprotein signal segments, Nicotiana nectarin signal peptide (U.S. Pat. No. 5,939,288), tobacco extensin signal, the soy oleosin oil body binding protein signal, Arabidopsis thaliana vacuolar basic chitinase signal peptide, as well as native signal sequences of the peptide of the invention. In addition, a nucleic acid molecule of the present invention can be joined to a fusion segment that directs the encoded peptide to the proteosome, such as a ubiquitin fusion segment. Recombinant molecules may also include intervening and/or untranslated sequences surrounding and/or within the nucleic acid sequences of nucleic acid molecules of the present invention.

### Host Cells

Another embodiment of the present invention includes a recombinant cell comprising a host cell transformed with one or more recombinant molecules of the present invention. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Although peptides discussed herein possess antifungal and antibacterial activity, suitable quantities of recombinant peptide of the invention can be obtained from bacterial or fungal host cells. More specifically, the peptide can be produced as a fusion protein, which is processed upon recovering the fusion protein from the recombinant host cell. An example of such a system is described by Hara and Yamakawa (1996) whereby B. mori moricin was produced as a fusion protein from E. coli. The fusion protein was harvested from the recombinant host cells and cleaved with cyanogen or o-iodosobenzoic acid to release the bioactive moricin peptide. A similar system could readily be devised to produce peptides of the present invention in bacterial or fungal host cells.

Suitable host cells to transform include any cell that can be transformed with a polynucleotide of the present invention. Host cells of the present invention either can be endogenously (i.e., naturally) capable of producing peptides of the present invention or can be capable of producing such peptides after being transformed with at least one polynucleotide molecule of the present invention. Host cells of the present invention can be any cell capable of producing at least one protein of the present invention, and include bacterial, fungal (including yeast), parasite, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; BHK cells; MDCK cells; CRFK cells; CV-1 cells; COS cells; Vero cells; and non-tumorigenic mouse myoblast G8 cells (e.g., ATCC CRL 1246). Additional appropriate mammalian cell hosts include other kidney cell lines, other fibroblast cell lines (e.g., human, murine or chicken embryo fibroblast cell lines), myeloma cell lines, Chinese hamster ovary cells, mouse NIH/3T3 cells, LMTK cells and/or HeLa cells. Particularly preferred host cells are plant cells such as those available from Deutsche SammItmg von Mikroorganismen and Zellkulturen GmbH (German Collection of Microorganisms and Cell Cultures).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotide molecules of the present invention to correspond to the codon usage of the host cell, and the deletion of sequences that destabilize transcripts.

### Transgenic Plants

The term “plant” refers to whole plants, plant organs (e.g. leaves, stems roots, etc), seeds, plant cells and the like. Plants contemplated for use in the practice of the present invention include both monocotyledons and dicotyledons. Preferably, the transgenic plant is a commercially useful crop plant. Target crops include, but are not limited to, the following: cereals (wheat, barley, rye, oats, rice, sorghum and related crops); beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape, mustard, poppy, olives, sunflowers, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as omamentals (flowers, shrubs, broad-leaved trees and evergreens, such as conifers). Particularly preferred crops include field peas, chickpeas, wheat and barley.

Transgenic plants, as defined in the context of the present invention include plants (as well as parts and cells of said plants) and their progeny which have been genetically modified using recombinant techniques to cause production of at least one peptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

A polynucleotide of the present invention may be expressed constitutively in the transgenic plants during all stages of development. Depending on the use of the plant or plant organs, the peptides may be expressed in a stage-specific manner. Furthermore, depending on the use—particularly where the plant may be prone to fungal infection, the polynucleotides may be expressed tissue-specifically.

Regulatory sequences which are known or are found to cause expression of a gene encoding a peptide of interest in plants may be used in the present invention. The choice of the regulatory sequences used depends on the target plant and/or target organ of interest. Such regulatory sequences may be obtained flow plants or plant viruses, or may be chemically synthesized. Such regulatory sequences are well known to those skilled in the art.

Other regulatory sequences such as terminator sequences and polyadenylation signals include any such sequence functioning as such in plants, the choice of which would be obvious to the skilled addressee. An example of such sequences is the 3′ flanking region of the nopaline synthase (nos) gene of Agrobacterium tumefaciens.

Several techniques are available for the introduction of an expression construct containing a nucleic acid sequence encoding a peptide of interest into the target plants. Such techniques include but are not limited to transformation of protoplasts using the calcium/polyethylene glycol method, electroporation and microinjection or (coated) particle bombardment. In addition to these so-called direct DNA transformation methods, transformation systems involving vectors are widely available, such as viral and bacterial vectors (e.g. from the genus Agrobacterium). After selection and/or screening, the protoplasts, cells or plant parts that have been transformed can be regenerated into whole plants, using methods known in the art. The choice of the transformation and/or regeneration techniques is not critical for this invention.

Examples of transgenic plants expressing antifungal peptides are described in Banzet et al. (2002) and EP 798381. In each case, the expression of the recombinant antifungal peptide resulted in the transgenic plant being resistant to fungal infections. Similar procedures as outlined in these documents can be used to produce peptides of the invention which confer resistance to fungal infections to the transgenic plant.

### Transgenic Non-Human Animals

Techniques for producing transgenic animals are well known in the art. A useful general textbook on this subject is Houdebine, Transgenic animals—Generation and Use (Harwood Academic, 1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention include “acceptable carriers”. An acceptable carrier is preferably any material that the animal, plant, plant or animal material, or environment (including soil and water samples) to be treated can tolerate. Examples of such acceptable carriers include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used.

Pharmaceutical compositions contain a therapeutically effective amount of an antifungal peptide of the invention. A therapeutically effective amount of an antifungal peptide can be readily determined according to methods known in the art. Pharmaceutical compositions are formulated to contain the therapeutically effective amount of an antifungal peptide and a pharmaceutically acceptable carrier appropriate for the route of administration (topical, gingival, intravenous, aerosol, local injection) as known to the art. For agricultural use, the composition comprises a therapeutically effective amount of a peptide of the invention and an agriculturally acceptable carrier suitable for the organism (e.g., plant) to be treated.

The phrase ‘pharmaceutically acceptable carrier’ refers to molecular entities and compositions that do not produce an allergic, toxic or otherwise adverse reaction when administered to an animal, particularly a mammal, and more particularly a human. Useful examples of pharmaceutically acceptable carriers or diluents include, but are not limited to, solvents, dispersion media, coatings, stabilizers, protective colloids, adhesives, thickeners, thixotropic agents, penetration agents, sequestering agents and isotonic and absorption delaying agents that do not affect the activity of the peptides of the invention. The proper fluidity can be maintained, for example, by the use of a coating, such as lecithin, by the maintenance of the required particle size in the case of dispersion and by the use of surfactants. More generally, the peptides of the invention can be combined with any non-toxic solid or liquid additive corresponding to the usual formulating techniques.

Liquid compositions of the invention include water-soluble concentrates, emulsifiable concentrates, emulsions, concentrated suspensions, aerosols, wettable powders (or powder for spraying), pastes and gels.

A peptide of the invention can also be used in the form of powders for dusting, and granules, in particular those obtained by extrusion, compacting, impregnation of a granular carrier or by granulation of a powder, and effervescent tablets or lozenges.

Surfactants may also form a component of various compositions. Surfactants can be an emulsifier, dispersant or wetting agent of ionic or nonionic type or a mixture of such surfactants. Examples include, but are not limited to, polyacrylic acid salts, lignosulfonic acid salts, phenolsulfonic or naphthalenesulfonic acid salts, polycondensates of ethylene oxide with fatty alcohols or with fatty acids or with fatty amines, substituted phenols (in particular alkyophenols or arylphenols), salts of sulfosuccinic acid esters, taurine derivatives (in particular alkyl taurates), polyoxyethylated phosphoric esters of alcohols or of phenols, fatty acid esters of polyols, derivatives containing sulfate, sulfonate and phosphate functions of the above compounds.

Depending on the specific conditions being treated and the targeting method selected, such agents may be formulated and administered systemically or locally. Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For agricultural compositions, natural or synthetic, organic or inorganic materials may be used with which the compound is combined in order to facilitate its application to the plant, to seeds or the soil. This carrier is thus generally inert and it should be agriculturally acceptable, in particular on the plant treated. The carrier can be solid (clays, natural or synthetic silicates, silica, resins, waxes, solid fertilizers, etc.) or liquid (water, alcohols, in particular butanol, etc.).

Exposure of a plant pathogen to an antifungal peptide may be achieved by applying to plant parts or to the soil or other growth medium surrounding the roots of the plants or to the seed of the plant before it is sown using standard agricultural techniques such as spraying. The peptide may be applied to plants or to the plant growth medium in the form of a composition comprising the peptide in admixture with a solid or liquid diluent and optionally various adjuvants such as surface-active agents. Solid compositions may be in the form of dispersible powders, granules, or grains.

The compositions of the present invention can also be used in numerous products including, but not limited to, disinfectant hand soaps, hypo-allergenic hand care creme, shampoo, face soap, laundry products, dish washing products (including a bar glass dip) bathroom cleaning products, dental products (e.g., mouthwash, dental adhesive, saliva injector filters, water filtration) and deodorizing products.

One embodiment of the present invention is a controlled release formulation that is capable of slowly releasing a peptide of the present invention into an animal, plant, animal or plant material, or the environment (including soil and water samples). As used herein, a controlled release formulation comprises a peptide of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

The effective concentration of the peptide, vector, or host cell within the composition can readily be determined experimentally, as will be understood by the skilled artisan.

Examples of compositions comprising antifungal peptides is provided in U.S. Pat. No. 6,331,522. Similar compositions comprising the peptides of the invention could readily be produced by the skilled addressee.

### Antibodies

The invention also provides antibodies to peptides of the invention or fragments thereof. The present invention further provides a process for the production of antibodies to peptides of the invention.

The term “antibody” as used in this invention includes intact molecules as well as fragments thereof, such as Fab, F(ab′)2, and Fv which are capable of binding the epitopic determinant. These antibody fragments retain some ability to selectively bind to a peptide of the invention, examples of which include, but are not limited to, the following:

(1) Fab, the fragment which contains a monovalent antigen-binding fragment of an antibody molecule can be produced by digestion of whole antibody with the enzyme main to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction; F(ab)2 is a dimer of two Fab′ fragments held together by two disulfide bonds;

(4) Fv, defined as a genetically engineered fragment containing the variable region of the light chain and the variable region of the heavy chain expressed as two chains;

(5) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific (see, for example, WO 94/07921 and WO 98/44001) and

(6) Single domain antibody, typically a variable heavy domain devoid of a light chain.

Furthermore, the antibodies and fragments thereof may be humanised antibodies, for example as described in EP-A-239400.

The term “binds specifically” refers to the ability of the antibody to bind to at least one protein/peptide of the present invention but not other known moricin-like peptides such as those mentioned in WO 2005/080423.

As used herein, the term “epitope” refers to a region of a peptide of the invention which is bound by the antibody. An epitope can be administered to an animal to generate antibodies against the epitope, however, antibodies of the present invention preferably specifically bind the epitope region in the context of the entire peptide.

If polyclonal antibodies are desired, a selected mammal (e.g., mouse, rabbit, goat, horse, etc.) is immunised with an immunogenic peptide. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides peptides of the invention or fragments thereof haptenised to another peptide for use as immunogens in animals.

Monoclonal antibodies directed against peptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies of the invention may be bound to a solid support and/or packaged into kits in a suitable container along with suitable reagents, controls, instructions and the like.

Preferably, antibodies of the present invention are delectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, e.g., biotin. Such labeled antibodies can be used in techniques known in the art to detect peptides of the invention.

### Uses

The peptides of the invention have many uses in medical, veterinary, agricultural, food preservative, household and industrial areas where it is desirable to reduce and/or prevent fungal or bacterial infections.

For instance, the peptides of the present invention can be used in pharmaceutical compositions to treat fungal infections, as well as bacterial infections (e.g., S. mutans, P. aeruginosa or P. gingivalis infections). Vaginal, urethral, mucosal, respiratory, skin, ear, oral, or ophthalmic fungal or bacterial infections that are amenable to peptide therapy include, but are not limited to: Candida albicans; Actinomyces actinomycetemcomitans; Actinomyces viscosus; Bacterlodesforsythus; Bacteriodesfragilis; Bacteriodes gracilis; Bacteriodes ureolyticus; Campylobacter concisus; Campylobacter rectus; Campylobacter showae; Campylobacter sputorum; Capnocytophaga gingivalts; Capnocytophaga ochracea; Capnocytophaga sputlgena; Clostridium histolydcum; Eikenella corrodens; Eubacterium nodatum; Fusobacterium nucleaium; Fusobacterium periodonticum; Peptostreptococcus micros; Porphyromonas endodontalls; Porphyrontonas gingivalis; Prevotella intermedia; Prevotella nigrescens; Propionibacterium acnes; Pseudomonas aeruginosa; Selenomonas noxia; Staphylococcus aureus; Streptococcus constellatus; Streptococcus gordonli; Streptococcus intermedius; Streptococcus mutons; Streptococcus oralis; Streptococcus pneumonia; Streptococcus sanguis; Treponema denticola; Treponema pectinovorum; Treponema socranslcii; Veillonellaparvula; and Wolinella succinogenes.

For agricultural applications, the antifungal peptide may be used to improve the disease-resistance or disease-tolerance of crops either during the life of the plant or for post-harvest crop protection. The growth of pathogens exposed to the peptides is inhibited. The antifungal peptide may eradicate a pathogen already established on the plant or may protect the plant from future pathogen attack. A pathogen may be any fungus growing on, in or near the plant. Improved resistance is defined as enhanced tolerance of the plant, or the crop after harvesting, to a fungal pathogen when compared to a wild-type plant. Resistance may vary from a slight decrease in the effects, to the total eradication so that the plant is unaffected by the presence of pathogen.

Thus, peptides of the invention can also be used to treat and/or prevent fungal infections of plants. Such plant fungi include, but are not limited to, those selected from the group consisting of the Genera: Alternaria; Asccchyta; Botrytis; Cercospora; Colletotrichum; Diplodia; Erysiphe; Fusarium; Leptosphaeria; Gaeumanomyces; Helminthosporium; Macrophomina; Nectria; Peronospora; Phoma; Phymatotrichum; Phytophthora; Plasmopara; Podosphaera; Puccinia; Puthium; Pyrenophora; Pyricularia; Pythium; Rhizoctonia; Scerotium; Sclerotinia; Septoria; Thielaviopsis; Uncinula; Venturia; and Verticillium. Specific examples of plant fungi infections which may be treated with the peptides of the present invention include, Erysiphe graminis in cereals, Erysiphe cichoracecrrum and Sphaerotheca fuligtnea in cucurbits, Podosphaera leucotricha in apples, Uncinula necator in vines, Puccinla sp. in cereals, Rhizoctonla sp. in cotton, potatoes, rice and lawns, Ustilago sp. in cereals and sugarcane, Venturia inaequalis (scab) in apples, Helminthosporium sp. in cereals, Septoria nodorum in wheat, Septoria triitici in wheat, Rhynchosporium secalis on barley, Botrytis cinerea (gray mold) in strawberries, tomatoes and grapes, Cercospora arachidicola in groundnuts, Peronospora tabacina in tobacco, or other Peronospora in various crops, Pseudocercosporella herpotricholdes in wheat and barley, Pyrenophera teres in barley, Pyricularla oryzae in rice, Phytophthora infestans in potatoes and tomatoes, Fusarium sp. (such as Fusarium oxysporum) and Verticillium sp. in various plants, Plasmopara viticola in grapes, Alternaria sp. in fruit and vegetables, Pseudoperonospora cubensis in cucumbers, Mycosphaerella fijiensis in banana, Ascochyta sp. in chickpeas, Leptosphaeria sp. on canola, and Colleotrichum sp. in various crops.

An antifungal peptide according to the invention may also be used as a preservative to maintain the freshness and shelf life of food products such as cheese, bread, cakes, meat, fish, preserves, feed for animals and the like. The antifungal peptide may also be used in antimicrobial food packaging such as coating plastics or polymers or incorporation within edible coating or films. For example peptide coatings and films can contain adequate amounts of antifungal peptide(s) for use on such products as cheese, sweets, dried goods and the like.

### EXAMPLES

### Example 1

### Peptide Purification

### Materials and Methods

### Insects

Galleria mellonella (wax moth) were reared on an artificial diet. Last instar larvae were injected with 10 μl of water containing approximately 10⁶ cells of each of Escherichia coli and Micrococcus luteus. As a control, some larvae were injected with 10 μl of phosphate buffered saline solution. Larvae were left at room temperature for 48 hours before extracting hemolymph by removal of a proleg. Hemolymph was collected on ice in a tube containing a few crystals of phenylthiourea, centrifuged for 5 min to remove cell debris, and frozen at −80° C.

### Antifungal and Antibacterial Activity Assays

Samples were tested for activity using an inhibition zone plate assay. For the bacteria E. coli and M. luteus, plates were prepared using nutrient agar (Oxoid) and a cell density of approximately 5×10⁶ cells/ml.

For fungi, plates were prepared using YPD broth (10 g/l yeast extract, 10 g/l peptone, 40 g/l D-glucose) containing 0.8% agarose and a spore density of approximately 10⁶ spores/ml. To test for activity, 2 μl of the sample of interest was spotted on the surface of the plate, and the organism grown under appropriate conditions (overnight at 37° C. for bacteria, 1-3 days at room temperature for fungi) until the presence or absence of clearance zones could be detected. The fungi tested were Fusarium graminearum, Fusarium oxysporum, Alternaria alternata, Ascochyta rabiei, Colletotrichum gloeosporioides, Leptosphaeria maculans and Aspergillus niger.

### Peptide Purification

Two crude hemolymph samples from different G. mellonella immunisations were processed separately by C18 solid phase extraction. The thawed hemolymph (1.8 ml or 4.8 ml) was diluted into an equal volume of 0.1% trifluoroacetic acid (TFA), and shaken on ice for 30-45 min. The samples were loaded onto C18 solid phase extraction cartridges (Maxi-Clean, 300 or 900 mg cartridges, Alltech). The cartridges were washed with 20% acetonitrile/0.05% TFA and eluted with 60% acetonitrile/0.05% TFA. Eluted samples were dried in a Speedvac (Savant) and resuspended in 100 μl water. The samples were tested against E. coli, M. luteus and various fungi using the plate assay described above. The resuspended hemolymph was loaded onto a Jupiter C18, 5 μm, 300 Å, 250×10 mm semi-prep column (Phenomenex) running on a System Gold HPLC (Beckman) monitoring absorbance at either 225 or 215 nm. The column was equilibrated in solvent A (2% acetonitrile, 0.065% TFA), and eluted with a gradient from 0-70% solvent B (95% acetonitrile, 0.05% TFA) over 70 min at 5 ml/min. Active fractions for all chromatography steps were selected by drying 30-500 μl of each fraction in a Speedvac, resuspending in 10 μl water, and testing for activity against F. graminearum. The active fractions from the semi-prep column were purified further by several steps of reverse phase chromatography.

For Gm-moricinD, the active fraction was diluted in an equal volume of 0.05% TFA and loaded onto a Prosphere C18, 5 μm, 300 Å, 250×4.6 mm column (Alltech) equilibrated in 10% solvent B on the HPLC. The column was eluted with a gradient of 15-55% B nmning over 60 min at 1 ml/min. The active fraction was then diluted in an equal volume of 0.05% TFA and loaded onto a μRPC C2/C18, 3 μm, 100×2.1 mm column (Amersham Biosciences). This column was equilibrated in solvent A running on a SMART system (Amersham Biosciences) and was eluted with a gradient of 0-100% solvent B running over 25 min at 200 μl/min while monitoring at 215, 254 and 280 nm.

Gm-moricinC3 was purified in a similar manner to Gm-moricinD, except that fractions from the C2/C18 column were tested directly against F. graminearum.

### Peptide Identification

The fractions of interest were analysed on a Voyager Elite MALDI-TOF mass spectrometer (Perseptive Biosystems) using 0.5 μl of sample plus 0.5 μl of matrix. For linear mode spectra the matrix was sinapinic acid and the standard was a mixture of cecropin A and myoglobin, and for reflector mode spectra the matrix was α-cyano-4-hydroxycinnamic acid and the standard was a tryptic digest of bovine serum albumin. For N-terminal amino acid sequencing the purified peptides were dried onto fibre glass disks and subject to Edman degradation using a Procise Model 492 Protein Sequencer (Applied Biosystems), in accordance with the manufacturers instructions.

### Results and Discussion

Two different batches of crude hemolymph were processed by C18 solid phase extraction and C18 semi-preparative chromatography. The samples obtained after partial purification by C18 solid phase extraction showed activity against E. coli, M. luteus, F. graminearum, A. alternata, A. rabiei, C. gloeosportoides, L. maculans and A. niger. Further purification of samples on a C18 semi-preparative column produced fractions eluting between approximately 25-40% acetonitrile that showed activity against the test organism F. graminearum. Two fractions from different positions in the gradient were purified further on a C18 analytical column.

For Gm-moricinC3, purification on the C18 analytical column resulted in two fractions that showed activity against F. graminearum. These fractions were pooled and purified further on a C2/C18 column, resulting in three fractions which had activity against F. graminearum. One of these fractions was judged sufficiently pure by mass spectroscopy for sequencing by Edman degradation.

For Gm-moricinD, purification on the C18 analytical column resulted in two fractions that showed activity against F. graminearum. One fraction was purified further on a C2/C18 column, resulting in two fractions which had activity against F. graminearum. One of these fractions was judged sufficiently pure by mass spectroscopy for sequencing by Edman degradation.

MALDI mass spectroscopy and Edman sequencing were used to identify the purified peptides. Gm-moricinC3 had an apparent molecular weight of 3923.0 Da and a partial amino acid sequence of KVPIGAIKKGGKIIKKGLGVIGAAGTAHEVYS (SEQ ID NO:23). Note that Gm-moricinC1 (residues 26 to 63 of SEQ ID NO:4, molecular mass 3932.3 Da) co-purified with Gm-moricinC3.

Gm-moricinD had an apparent molecular mass of 3832.8 Da and a partial amino acid sequence of KGIGSALKKGGKIIKGGLGALGAIGTGQQVYE (SEQ ID NO:24). Searches of the non-redundant databases using BLASTP for short matches indicated that these two peptides had some similarity to the known peptides moricin from Bombyx mori and other Lepidoptera.

### Example 2

### Identification of cDNAs Encodins G. mellonella Morkin-Like Peptides

### Preparation of Total RNA and Poly(A)⁺ RNA

Fat body tissue was dissected from G. mellonella larvae at 24 hours after injection with E. coli and M. luteus cell suspension. Larvae that had been chilled on ice for at least 30 min were pinned in a Sylgard dish under ice-cold PBS and opened by a longitudinal incision down the dorsal midline. The gut was removed and fat body was collected with fine watch-makers forceps. Dissected fat body was briefly blotted on absorbent tissue and snap-frozen in a microfuge tube held in liquid nitrogen. The frozen tissues were stored at −80° C.

Total RNA was isolated using Trizol reagent (Astral Scientific). Briefly, approximately 500 mg of frozen fat body tissue was resuspended in 1 mL of Trizol reagent and homogenised in a Polytron tissue homogeniser.

Polyadenylated RNA was isolated by two rounds of selection on oligo(dT)-cellulose spun-column chromatography using the mRNA purification kit (Amersham Biosciences). Approximately 1 mg of total RNA was bound to an oligo(dT)-cellulose spin column, washed and eluted in 1 mL of low salt buffer according to the manufacturer's instructions. The eluted RNA was bound to a second spin column, washed and eluted as described above in a final volume of 1 mL. The mRNA was precipitated by addition of sodium acetate to a final concentration of 0.1M with 200 μL ethanol. The mRNA was recovered by centrifugation and resuspended in 5 μL of DEPC-treated water.

Preparation of a cDNA Library

A cDNA library was prepared from approximately 5 μg of mRNA using a Lambda UniZap cDNA synthesis and cloning system (Stratagene). Purified cDNA (approx. 20 ng) was ligated to 1 μg of vector DNA and packaged with Gigapack® III Gold packaging extract (Stratagene) to yield a cDNA library with a titre of 5×10⁵ plaque forming units per mL.

Identification of Gm-moricinC4, Gm-moricinC5 and Gm-moricinD by PCR on the cDNA Library

The oligonucleotide sequences for Gm-moricinC3-C5 and Gm-moricinD were determined by amplifying sequences from the cDNA library by PCR with degenerate primers followed by PCR with specific primers. Primer sequences are shown in Table 2.

TABLE 2
Primer sequences used to isolate moricin genes in Galleria mellonella.

| Primer name | Primer sequence |
|---|---|
| GmC3R5 | 5′-GCTTTACCACCCTTTTTGATG-3′ (SEQ ID NO: 53) |
| GmC3F3 | 5′-GGTTTGGGTGTGGTAGGTG-3′ (SEQ ID NO: 54) |
| GmC3R5r | 5′-CATCAAAAAGGGTGGTAAAGC-3′ (SEQ ID NO: 55) |
| GmC3F3r | 5′-CACCTACCACACCCAAACC-3′ (SEQ ID NO: 56) |
| GmC3utr5 | 5′-ACAGTCGCAGTCATTCTCAGTC-3′ (SEQ ID NO :57) |
| GmC3utr3 | 5′-CGTAGCCAATAATAATACTCCACA-3′ (SEQ ID NO: 58) |
| GmC3u1f | 5′-ACCTTCACTCCTTGCTATCA-3′ (SEQ ID NO: 59) |
| GmC3u13f | 5′-TAACTTACTTTTCACTTCCA-3′ (SEQ ID NO: 60) |
| GmC3u2r | 5′-ACTTATATATATATATATCG-3′ (SEQ ID NO: 61) |
| GmC3u4r | 5′-AAACTTATATAAATATATCG-3′ (SEQ ID NO: 62) |
| GmD-1 | 5′-CCNAARGGNATCGGNWSTGC-3′ (SEQ ID NO: 63) |
| GmD-R | 5′-TCRTANACYTGYTGNCCNGT-3′ (SEQ ID NO: 64) |
| GmDF3 | 5′-CAAGAAAGGCGGCAAAATTA-3′ (SEQ ID NO: 65) |
| GmDR5 | 5′-ACCGATGGCTCCTAATGCT-3′ (SEQ ID NO: 66) |
| GmDutr5 | 5′-TGAATTAAAACCTAATAAAC-3′ (SEQ ID NO: 67) |
| GmDutr3 | 5′-TATTTGAGACAACTGGCTG-3′ (SEQ ID NO: 68) |
| GmDint5 | 5′-CTCAAGAAAGGCGGCAAAAT-3′ (SEQ ID NO: 69) |
| GmDR5 | 5′-ACCGATGGCTCCTAATGCT-3′ (SEQ ID NO: 70) |
| GmCint5 | 5′-GGTCAAGCCGACCCTAAGGTGCC-3′ (SEQ ID NO: 71) |
| GmCint3 | 5′-GGCTATATACTTCAGTGCGCTGT-3′(SEQ ID NO: 72) |
| GmDint3ex | 5′-ATAGTCGAGAAATGGCAAAAT-3′ (SEQ ID NO: 73) |
| GmDint5ex | 5′-CTGCGCTATCGGCATACACTA-3′ (SEQ ID NO: 74) |

For Gm-moricinD, degenerate primers (GmD-1, GmD-R) designed from the partial amino acid sequence of the peptide were first used to amplify a product from the cDNA library by PCR. Primers designed from this sequence (GmDF3, GinDRS) were used in nested PCR with vector primers to determine the 5′ and 3′ regions of the gene. A third set of primers specific to the 5′ and 3′ untranslated regions (GmDutr5, GmDutr3) were then designed and used to determine the complete open reading frame.

Gm-moricinC3 was found by nested PCR on the cDNA library using specific primer pairs (CanC3R5, GmC3R5r, GmC3F3, GmC3F3r) designed from sequences obtained when searching for introns (see below) and vector primers to determine the 5′ and 3′ regions of the gene. The full-length sequence was then obtained by PCR using primers specific to the 5′ and 3′ untranslated regions (GmC3utr5, GmC3utr3). Gm-moricinC4 and C5 were found by nested PCR with primers designed from the untranslated region of previously identified PCR products (CrinC3u1f, GmC3u2r, GmC3u1f, GmC3u4r, GmC3u13f) and vector primers.

To detect introns in the Gm-moricin genes, genomic DNA was isolated from G. mellonella using the Quantum Prep Aquapure Genomic DNA kit (Bio-Rad). Primer pairs designed to anneal to the 5′ and 3′ regions of the genes (GmCint5, GmCint3; GmDint5, GmDR5) were used in two-step PCR reactions on genomic DNA or the cDNA library pools. Reaction products were purified and ligated into pGEM-T Easy (Promega). For Gm-moricinD, extra internal primers (GmDint5ex, GmDint3ex) were used to obtain the full intron sequence.

### Results and Discussion

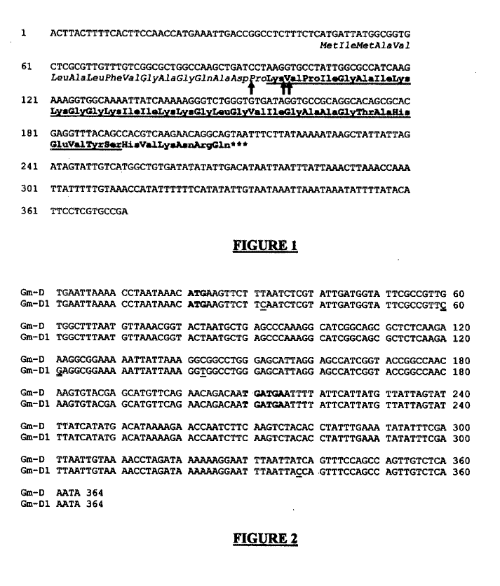

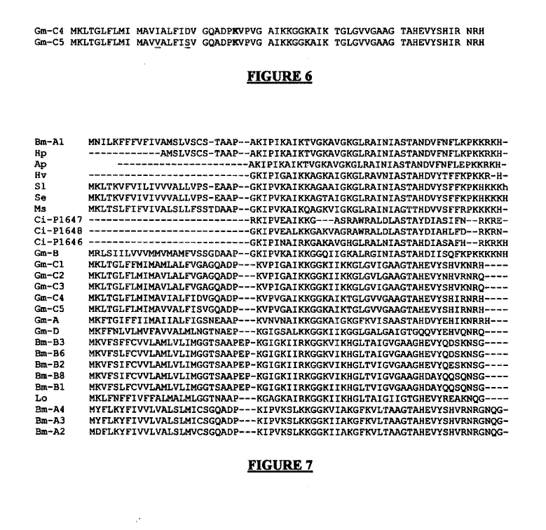

For Gm-moricinC3 and Gm-moricinD, the partial amino acid sequences determined by Edman degradation were identical to sections of the translated nucleotide sequences isolated from the G. mellonella fat body cDNA library (FIGS. 1 to 4). This allowed extraction of the predicted open reading frames for these peptides from the corresponding nucleotide sequences. For Gm-moricinC4 and Gm-moricinC5, nucleotide sequences were obtained by PCR on the G. mellonella fat body cDNA library (FIG. 5). Amino acid sequences were translated from the predicted open reading frames of these nucleotide sequences. The analysis of intron data was then critical for distinguishing between independent genes and allelic variants of the various moricins. Single introns were identified by PCR for Gm-moricinC4 (347 bp), Gm-moricinC5 (336 bp), and Gm-moricinD (1072 bp), but not for Gm-moricinC3. The introns all occurred at the same position in the mature amino acid sequence, which is after residue 14. The introns of Gm-moricinC4 and Gm-moricinC5 differed by 17 nucleotides.

Correlation of the nucleotide, amino acid and intron sequences with the mass spectroscopy data allowed determination of the complete sequences of Gm-moricinC3 (FIG. 1), Gm-moricinC4, Gm-moricinC5 (FIGS. 5 and 6) and Gm-moricinD (FIGS. 2 to 4). The full-length peptides are 63 residues long and the mature peptides in G. mellonella start at residue 26 following cleavage after the sequence ADP or AEP. This processing is consistent with predictions made by SignalP and knowledge of other insect antimicrobial peptides (Boman, et al., 1989). A ClustalW alignment of all currently known moricins is shown in FIG. 7. Construction of a phylogenetic tree based on the alignment of the mature peptide sequences (FIG. 7) indicate that the Gm-moricinC1-C5 peptides are all closely related. Gm-moricinD clusters with the L. oblique moricin transcript and the B. mori moricinB1-B8 peptides.

For Gm-moricinC3, no allelic variants were identified. The closest known relatives of Gm-moricinC3 are Gm-moricinC1 and Gm-moricinC2. Mature Gm-moricinC3 is 97% and 92% identical to Gm-moricinC1 and Gm-moricinC2, respectively.

For Gm-moricinC4 and Gm-moricinC5, the nucleotide sequences differ by only 4 bases (2%) (FIG. 5), and the mature amino acid sequences are identical (FIG. 6). However, Gm-moricinC4 and Gm-moricinC5 have been classified as separate genes due to their introns differing by 17 nucleotides. Although not isolated as a peptide, the mature Gm-moricinC4 and Gm-moricinC5 sequence was shown to be expressed by the LC/MS detection of protease fragments in G. mellonella hemolymph. Mature Gm-moricinC4 and Gm-moricinC5 are 84% identical to Gm-moricinCl and 81% identical to Gm-moricinC2, and are unique in the moricin family for having a threonine residue at position 16 in the mature sequence (FIG. 7).

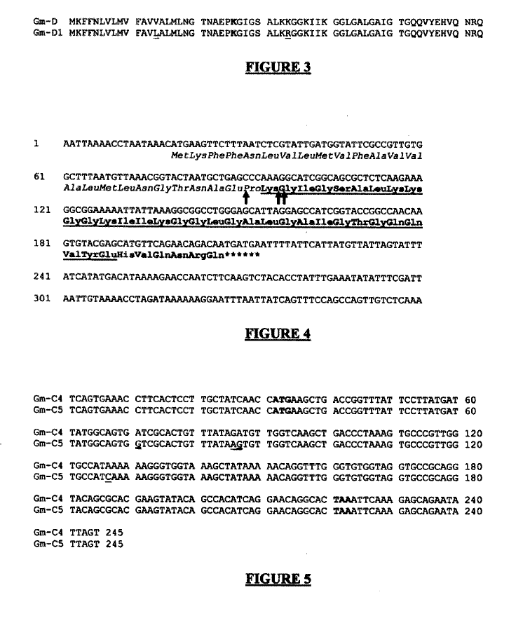

For Gm-moricinD, PCR experiments identified two distinct sequences which are likely to be allelic variants (FIGS. 2 and 3). One of these matched the experimentally determined amino acid sequence (Gm-moricinD), and the other (Gm-moricinD1) differed by only five nucleotide substitutions (2.6%). Two of these differences were in the peptide open reading frame and resulted in two changed amino acids (V14L, K34R) (FIG. 3). Mature Gm-moricinD is 57 and 63% identical to Gm-moricinC1 and Gm-moricinC2, respectively. Outside of G. mellonella, Gm-moricinD has 57% identity to the translated sequence of an unannotated L. obliqua transcript and 44-47% identity to the moricinB1-B8 peptides from B. mori (Cheng, et al., 2006). The L. obliqua moricin has only been identified as an EST and has not been studied as a peptide. No evidence has been found for expression of the B. mori moricin131-B8 peptides, either by RT-PCR (Cheng, et al., 2006) or the presence of transcripts in the EST libraries. Within the subgroup of moricins which includes the L. obliqua transcript and the B. mori moricinB1-B8 peptides, Gm-moricinD is the first peptide to be isolated and shown to have any activity, specifically antifungal activity.

### Example 3

### Activity of Synthetic a G. mellonella Gm-moricinD Against Various Fungi

Gm-moricinD (SEQ ID NO:7) was synthesised by Auspep (Melbourne, Australia) using standard peptide synthesis techniques. The peptide was tested for activity against the bacteria E. coli and M. luteus, and against spores of the fungi F. graminearum, F. oxysponan, A. rabiei and L. maculans generally as described in Example 1. The concentrations tested were 0.1, 1, 10 and 100 μM, and 1 μg/μl. Gm-moricinD showed no activity against E. coli or M. luteus at 1 μg/μl, but showed activity against spores of F. graminearum at the 10 μM level, and spores of L. macularis, F. oxysporum and A. rabiei at the 100 μM level. The demonstration of antifungal activity for Gm-moricinD is the first evidence of any functional role of moricin peptides in the sub-group consisting of Gm-moricinD, B. mori moricinB1-B8 and L. obliqua moricin.

### Example 4

### Activity of Synthetic G. Mellonella Gm-moricinC3 and Gm-moricinc4/C5 Against Various Fund

Gm-moricinC3 (SEQ ID NO:5) and Gm-moricinC4/C5 (SEQ ID NO:1 and SEQ ID NO:3) were synthesised by Auspep (Melbourne, Australia) using standard peptide synthesis techniques. The peptides were tested for activity against the bacteria E. coli and M. luteus, and against spores of the fungi F. gramineanan, F. oxysporum and L. maculans as described in Example I. The concentrations tested were 0.1, 1, 10 and 100 μM, and 1 μg/μl. Gm-moricinC3 showed activity against the bacteria E. coli and M. luteus and spores of the fungi F. oxysporum and L. maculans at 100 μM and activity against spores of the fungus F. graminearum at the 10 μM level. The Gm-moricinC4/C5 peptide showed activity against the gram-negative bacterium E. coli at 100 μM, but no activity against the gram-positive bacterium M. luteus at the concentrations tested up to 1 μg/μl. Gm-moricinC4/C5 peptide was active against spores of the fungi F. graminearum and L. maculans at 100 μM, but showed no activity against spores of the fungus F. oxysporum.

### Example 5

### Expression of Antifungal Peptides in Arabidopsis

Agrobacterium-Mediated Transformation of Arabidopsis with the G. Mellonella Gm-moricinD Gene

DNA encoding Gm-moricinD is cloned into the Agrobacterium transfer vector, p277 (obtained from CSIRO Plant Industry, Can berra, Australia). This vector was constructed by insetting the Nod frag from pART7 into pART27 (Gleave, 1992). The p277 vector contains the CaMV 35S promoter and OCS terminator for plant expression, markers for antibiotic selection, and the sequences required for plant transformation. Gm-moricinD DNA constructs are chosen for transformation into Arabidopsis thaliana—the mature Gm-moricinD with no signal peptide, the full-length Gm-moricinD including its native signal peptide, and a fusion consisting of an Arabidopsis vacuolar basic chitinase signal peptide and the mature Gm-moricinD sequence. These constructs were synthesised by PCR and directionally cloned into the p277 transfer plasmid.

Transformation of the Agrobacterium strain GV3101 is achieved using the triparental mating method. This involved co-streaking cultures of A. tumefasciens GV3101, E. coli carrying a helper plasmid, RK2013, and E. coli carrying the desired recombinant p277 plasmid onto a non-selective LB plate. Overnight incubation at 28° C. results in a mixed culture which is collected and dilution streaked onto LB plates which selected for A. tumefasciens GV3101 carrying the p277 recombinant plasmid.

Arabidopsis plants are cultured by standard methods at 23° C. with an 18 hr light period per day. Transformation of Arabidopsis plants is carried out by floral dipping. Plants are grown to an age, 3-5 weeks, where there will be many flower stems presenting flowers at various stages of development. An overnight culture of transformed A. tumefasciens GV3101 is pelleted and resuspended in 5% sucrose containing the wetting agent Silwet-77. Flowers are dipped into the bacterial suspension and thoroughly wetted by using a sweeping motion. The plants are wrapped in plastic film and left overnight on a bench top at room temperature, before being unwrapped and placed back into a plant growth cabinet maintained at 21° C. The dipping is repeated 1-2 weeks later to increase the number of transformed seeds. The seeds are collected 3-4 weeks after dipping, dried in seed envelopes for the appropriate length of time for each ecotype, then sterilised and germinated on Noble agar plates containing selective antibiotics and an antifungal agent.

Positive transformants are transplanted into Arasystem pots (Betatech), grown to maturity inside Aracon system sleeves and the seeds carefully collected. Transformed Arabidopsis plants (T1 generation) are screened by PCR to confirm the presence of the recombinant gene. Genomic DNA is extracted from the leaves of plants transformed with the full-length Gm-moricinD construct using the Extract-N-Amp Plant PCR and Extract-N-Amp Reagent kits (Sigma). PCR on the extracts is performed using primers specific to the Gm-moricinD gene.

T1 seedlings can be transplanted and cultivated for seed through two generations to eventually isolate the homozygous T3 seeds. T3 plants can then be screened for increased resistance to fungal disegse (see below). T3 plants can also be screened by reverse-transcriptase PCR(RT-PCR) to confirm the expression of the recombinant gene. Plants transformed with the full-length Gm-moricinD construct are randomly selected for analysis. Leaves from these plants are snap frozen and ground in liquid nitrogen using a mortar and pestle. RNA is isolated using the RNeasy Plant kit (Qiagen). cDNA is prepared from the RNA using the iScript cDNA Synthesis kit (Bio-Rad). PCR is performed using 1 μl of cDNA, recombinant Taq polymerase (Invitrogen), an annealing temperature of 54° C., and Gm-moricinD specific primers. 3 μl of each 25 PCR reaction is visualised on a 1.2% agarose gel.

Inoculation Protocol Using Fusarium oxysporum

A Fusarium oxysporum strain known to be pathogenic to Arabidopsis was obtained from J. Manners (CSIRO Plant Industry, Queensland, Australia). The fungal isolate can be maintained on ½ strength Potato Dextrose Agar (PDA).

From maintenance stocks, cores are taken and used to inoculate 500 ml Potato Dextrose Broth (PDB). Flasks are incubated on a shaker for 7 days at 28° C. The inocilum is drained through miracloth prior to quantification with a haemocytometer. The spores are diluted with sterile distilled water and used to inoculate Arabidopsis strain's.

Several ecotypes of Arabidopsis are cultivated for testing, including Columbia 0 (Col-0), Landsberg erecta (L-er) and Sg-1 (obtained from CSIRO Plant Industry, Canberra, Australia). Arabidopsis plants used in the inoculation are grown singly in ‘jiffy’ pots for approximately 2-3 weeks. Watering of plants is ceased approximately 4 days prior to infection. Arabidopsis plants are inoculated by adding 5 ml of resuspended spores directly onto the soil near the plant stem to give a total dose of 4×10⁵-2×10⁶ spores. Plants are incubated at 25° C. and scored for wilt symptoms and/or death over 14 days post inoculation.

To further characterize the level of disease caused to a specific genotype, a set of oligonucleotide primers (see Example 4 of WO 2005/080423) is used to amplify a region of 18S rRNA from F. oxysporum. The primers demonstrate little to no homology with Arabidopsis RNA and act to indicate the difference in fungal RNA levels as compared to the amount of plant RNA.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

The present application claims priority from AU 2007901600, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Banzet, N. et al. (2002) Plant Sci., 162; 995-1006.
Boman, H. G. et al. (1989) J. Biol. Chem., 264; 5852-5860.
Cheng, T. et al. (2006) Genomics, 87; 356-365.
DeLucca, A. J., and Walsh, T. J. (1999) Antimicrob. Agents Chemother., 43; 1-11.
Gleave, A. P. (1992) Plant Mol. Biol., 20; 1203-1207.
Hara, S, and Yamakawa, M. (1995) 1 Biol. Chem., 270; 29923-29927.
Hara, S, and Yamakawa, M. (1996) Biochem. Biophys. Res. Commun., 220; 664-669.
Harayama, S. (1998) Trends Biotech., 16; 76-82.
Hemmi, H., Ishibashi, J., Hara, S, and Yamakawa, M. (2002) FEES Letters, 518; 33-38.
Needleman, S. B. and Wunsch, C. D. (1970) J. Mol. Biol., 48; 443-453.

1. A substantially purified peptide which comprises a sequence selected from the group consisting of:
i) an amino acid sequence as provided in SEQ ID NO:1 and SEQ ID NO:3,
ii) an amino acid sequence which is at least 85% identical to SEQ ID NO:1 and/or SEQ ID NO:3,
iii) an amino acid sequence as provided in SEQ ID NO:5,
iv) an amino acid sequence which is at least 98% identical to SEQ ID NO:5,
v) an amino acid sequence as provided in SEQ ID NO:7 or SEQ ID NO:9,
vi) an amino acid sequence which is at least 64% identical to SEQ ID NO:7 and/or SEQ ID NO:9,
vii) a biologically active fragment of any one of i) to vi), and
viii) a precursor comprising the amino acid sequence according to any one of i) to vii),
wherein the peptide, or fragment thereof, has antifungal and/or antibacterial activity.

2-3. (canceled)

4. The peptide of claim 1, wherein the peptide has antifungal activity against a fungus selected from the group consisting of Fusarium graminearum, Fusarium oxysporum, Ascochyta rabiei, and Leptosphaeria maculans.

5. The peptide of claim 1 which is fused to at least one other polypeptide/peptide sequence.

6. An isolated polynucleotide comprising a sequence selected from the group consisting of:
i) a sequence of nucleotides provided in any one of SEQ ID NOS: 11 to 20;
ii) a sequence encoding a peptide of claim 1;
iii) a sequence of nucleotides which is at least 85% identical to at least one of SEQ ID NOS: 11 to 14;
iv) a sequence of nucleotides which is at least 98% identical to SEQ ID NO:15 or SEQ ID NO:16;
v) a sequence of nucleotides which is at least 64% identical to at least one of SEQ ID NOS: 17 to 20; and
vi) a sequence which hybridizes to any one of (i) to (v) under high stringency conditions.

7. (canceled)

8. A vector comprising the polynucleotide of claim 6.

9. A host cell comprising the vector of claim 8.

10. The host cell of claim 9 which is a plant cell.

11. A process for preparing a peptide comprising cultivating a host cell according to claim 9 under conditions which allow expression of the polynucleotide encoding the peptide, and recovering the expressed peptide.

12. An antibody which specifically binds a peptide of claim 1.

13. A composition comprising a peptide of claim 1, and one or more acceptable carriers.

14. A method for killing, or inhibiting the growth and/or reproduction of a fungus and/or a bacteria, the method comprising exposing the fungus and/or bacteria to a peptide of claim 1.

15. A transgenic plant, the plant having been transformed with a polynucleotide according to claim 6, wherein the plant produces a peptide encoded by the polynucleotide.

16. A method of controlling fungal and/or bacterial infections of a crop, the method comprising cultivating a crop of transgenic plants of claim 15.

17. A transgenic non-human animal, the animal having been transformed with a polynucleotide according to claim 6, wherein the animal produces a peptide encoded by the polynucleotide.

18. A method of treating or preventing a fungal and/or bacterial infection in a patient, the method comprising administering to the patient a peptide of claim 1.

19. (canceled)

20. A kit comprising a peptide of claim 1.

21. A method for killing, or inhibiting the growth and/or reproduction of a fungus, the method comprising exposing the fungus to a peptide which comprises a sequence selected from the group consisting of:
i) an amino acid sequence comprising residues 28 to 65 of any one of SEQ ID NOS: 44 to 48,
ii) an amino acid sequence comprising residues 26 to 63 of SEQ ID NO:49,
iii) an amino acid sequence comprising residues 26 to 66 of any one of SEQ ID NOS: 50 to 52,
iv) an amino acid sequence which is at least 50% identical to any one of i) to iii), and
v) a biologically active fragment of any one of i) to iv).

22. A method of controlling fungal infections of a crop, the method comprising cultivating a crop of transgenic plants which produce a peptide which comprises a sequence selected from the group consisting of:
i) an amino acid sequence comprising residues 28 to 65 of any one of SEQ ID NOS: 44 to 48,
ii) an amino acid sequence comprising residues 26 to 63 of SEQ ID NO:49,
iii) an amino acid sequence comprising residues 26 to 66 of any one of SEQ ID NOS: 50 to 52,
v) an amino acid sequence which is at least 50% identical to any one of i) to iii), and
v) a biologically active fragment of any one of i) to iv).

23-24. (canceled)

25. A composition comprising a polynucleotide of claim 6, and one or more acceptable carriers.
